(19) United States
(12) Patent Application Publication
(10) Pub. No.: US 2010/0041734 A1
(43) Pub. Date: Feb. 18, 2010

(54) MODULATION OF TOLL-LIKE RECEPTOR 7 EXPRESSION BY ANTISENSE OLIGONUCLEOTIDES

(75) Inventors: Ekambar Kandimalla, Southboro, MA (US); Mallikarjuna Putta, Burlington, MA (US); Lakshmi Bhagat, Framingham, MA (US); Daqing Wang, Bedford, MA (US); Dong Yu, Westboro, MA (US); FuGang Zhu, Bedford, MA (US); Sudhir Agrawal, Shrewsbury, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Idera Pharmaceuticals, Inc.

(21) Appl. No.: 12/534,462

(22) Filed: Aug. 3, 2009

Related U.S. Application Data
(60) Provisional application No. 61/086,011, filed on Aug. 4, 2008.

(51) Int. Cl. A61K 31/7105; C07H 21/02; A61P 37/04; A61P 31/04; A61P 33/02; A61P 33/06

(52) U.S. Cl. 514/44 A; 536/23.1

(57) ABSTRACT

Antisense oligonucleotide compounds, compositions and methods are provided for down regulating the expression of TLR7. The compositions comprise antisense oligonucleotides targeted to nucleic acids encoding TLR7. The compositions may also comprise antisense oligonucleotides targeted to nucleic acids encoding TLR7 in combination with other therapeutic and/or prophylactic compounds and/or compositions. Methods of using these compounds and compositions for down-regulating TLR7 expression and for prevention or treatment of diseases wherein modulation of TLR7 expression would be beneficial are provided.

### RELATED APPLICATIONS

This application claims the benefit of prior U.S. Provisional Patent Application Ser. No. 61/086,011, filed on Aug. 4, 2008, the contents of which are incorporated by reference in its entirety.

### BACKGROUND OF THE INVENTION

1. Field of the Invention

The present invention relates to Toll-Like Receptor 7 (TLR7). In particular, the invention relates to antisense oligonucleotides that specifically hybridize with nucleic acids encoding TLR7, thus modulating TLR7 expression and activity, and their use in treating or preventing diseases associated with TLR7 or wherein modulation of TLR7 expression would be beneficial.

2. Summary of the Related Art

Toll-like receptors (TLRs) are present on many cells of the immune system and have been shown to be involved in the innate immune response (Hornung, V. et al., (2002) J. Immunol. 168:4531-4537). TLRs are a key means by which mammals recognize and mount an immune response to foreign molecules and also provide a means by which the innate and adaptive immune responses are linked (Akira, S. et al. (2001) Nature Immunol. 2:675-680; Medzhitov, R. (2001) Nature Rev. Immunol. 1:135-145). In mammals, this family consists of at least 11 proteins called TLR1 to TLR11, which are known to recognize pathogen associated molecular patterns (PAMP) from bacteria, fungi, parasites and viruses and induce an immune response mediated by a number of transcription factors.

Some TLRs are located on the cell surface to detect and initiate a response to extracellular pathogens and other TLRs are located inside the cell to detect and initiate a response to intracellular pathogens. Table 1 provides a representation of TLRs, the known agonists therefore and the cell types known to contain the TLR (Diebold, S. S. et al. (2004) Science 303:1529-1531; Liew, F. et al. (2005) Nature 5:446-458; Hemmi H et al. (2002) Nat Immunol 3:196-200; Jurk M et al., (2002) Nat Immunol 3:499; Lee J et al. (2003) Proc. Natl. Acad. Sci. USA 100:6646-6651); (Alexopoulou, L. (2001) Nature 413:732-738).

TABLE 1

| TLR | | Cell Types Containing |
|---|---|---|
| Molecule | Agonist | Receptor |
| Cell Surface | | |
| TLRs: | | |
| TLR2 | bacterial lipopeptides | Monocytes/macrophages |
| | | Myeloid dendritic cells |
| | | Mast cells |
| TLR4 | gram negative bacteria | Monocytes/macrophages |
| | | Myeloid dendritic cells |
| | | Mast cells |
| | | Intestinal epithelium |
| TLR5 | motile bacteria | Monocyte/macrophages |
| | | Dendritic cells |
| | | Intestinal epithelium |
| TLR6 | gram positive bacteria | Monocytes/macrophages |
| | | Mast cells |
| | | B lymphocytes |
| Endosomal | | |
| TLRs: | | |
| TLR3 | double stranded RNA viruses | Dendritic cells |
| | | B lymphocytes |
| TLR7 | single stranded RNA viruses; | Monocytes/macrophages |
| | RNA-immunoglobulin | Plasmacytoid dendritic cells |
| | complexes | B lymphocytes |
| TLR8 | single stranded RNA viruses; | Monocytes/macrophages |
| | RNA-immunoglobulin | Dendritic cells |
| | complexes | Mast cells |
| TLR9 | DNA containing unmethylated | Monocytes/macrophages |
| | “CpG” motifs; DNA- | Plasmacytoid dendritic cells |
| | immunoglobulin complexes | B lymphocytes |

The signal transduction pathway mediated by the interaction between a ligand and a TLR is shared among most members of the TLR family and involves a toll/IL-1 receptor (TIR domain), the myeloid differentiation marker 88 (MyD88), IL-1R-associated kinase (IRAK), interferon regulating factor (IRF), TNF-receptor-associated factor (TRAF), TGFβ-activated kinasel, IκB kinases, IκB, and NF-κB (see for example: Akira, S. (2003) J. Biol. Chem. 278:38105 and Geller at al. (2008) Curr. Drug Dev. Tech. 5:29-38). More specifically, for TLRs 1, 2, 4, 5, 6, 7, 8, 9 and 11, this signaling cascade begins with a PAMP ligand interacting with and activating the membrane-bound TLR, which exists as a homo-dimer in the endosomal membrane or the cell surface. Following activation, the receptor undergoes a conformational change to allow recruitment of the TIR domain containing protein MyD88, which is an adapter protein that is common to all TLR signaling pathways except TLR3. MyD88 recruits IRAK4, which phosphorylates and activates IRAK1. The activated IRAK1 binds with TRAF6, which catalyzes the addition of polyubiquitin onto TRAF6. The addition of ubiquitin activates the TAK/TAB complex, which in turn phosphorylates IRFs, resulting in NF-kB release and transport to the nucleus. NF-kB in the nucleus induces the expression of proinflammatory genes (see for example, Trinchieri and Sher (2007) Nat. Rev. Immunol. 7:179-190).

The selective localization of TLRs and the signaling generated therefrom, provides some insight into their role in the immune response. The immune response involves both an innate and an adaptive response based upon the subset of cells involved in the response. For example, the T helper (Th) cells involved in classical cell-mediated functions such as delayed-type hypersensitivity and activation of cytotoxic T lymphocytes (CTLs) are Th1 cells. This response is the body's innate response to antigen (e.g. viral infections, intracellular pathogens, and tumor cells), and results in a secretion of IFN-gamma and a concomitant activation of CTLs.

As a result of their involvement in regulating an inflammatory response, TLRs have been shown to play a role in the pathogenesis of many diseases, including autoimmunity, infectious disease and inflammation (Papadimitraki et al. (2007) J. Autoimmun. 29: 310-318; Sun et al. (2007) Inflam. Allergy Drug Targets 6:223-235; Diebold (2008) Adv. Drug Deliv. Rev. 60:813-823; Cook, D. N. et al. (2004) Nature Immunol. 5:975-979; Tse and Horner (2008) Semin. Immunopathol. 30:53-62; Tobias & Curtiss (2008) Semin. Immunopathol. 30:23-27; Ropert et al. (2008) Semin. Immunopathol. 30:41-51; Lee et al. (2008) Semin. Immunopathol. 30:3-9; Gao et al. (2008) Semin. Immunopathol. 30:29-40; Vijay-Kumar et al. (2008) Semin. Immunopathol. 30:11-21). While activation of TLRs is involved in mounting an immune response, an uncontrolled or undesired stimulation of the immune system through TLRs may exacerbate certain diseases in immune compromised subjects or may cause unwanted immune stimulation. Thus, down-regulating TLR expression and/or activity may provide a useful means for disease intervention.

To date, investigative strategies aimed selectively at inhibiting TLR activity have involved small molecules (WO/2005/007672), antibodies (see for example: Duffy, K. et al. (2007) Cell Immunol. 248:103-114), catalytic RNAi technologies (e.g. small inhibitory RNAs), certain antisense molecules (Caricilli et al. (2008) J. Endocrinology 199:399), and competitive inhibition with modified or methylated or modified oligonucleotides (see for example: Kandimalla et al. US2008/0089883; Barrat and Coffman (2008) Immunol. Rev. 223:271-283). For example, chloroquine and hydroxylchloroquine have been shown to block endosomal-TLR signaling by down-regulating the maturation of endosomes (Krieg, A. M. (2002) Annu. Rev. Immunol. 20:709). Also, Huang et al. have shown the use of TLR4 siRNA to reverse the tumor-mediated suppression of T cell proliferation and natural killer cell activity (Huang et al. (2005) Cancer Res. 65:5009-5014), and the use of TLR9 siRNA to prevent bacterial-induced inflammation of the eye (Huang et al. (2005) Invest. Opthal. Vis. Sci. 46:4209-4216).

Additionally, several groups have used synthetic oligodeoxynucleotides having two triplet sequences, a proximal “CCT” triplet and a distal “GGG” triplet, a poly “G” (e.g. “GGGG” or “GGG”) or “GC” sequences that interact with certain intracellular proteins, resulting in the inhibition of TLR signaling and the concomitant production and release of pro-inflammatory cytokines (see for example: Lenert, P. et al. DNA Cell Biol. 22(10):621-631 (2003); Patole, P. et al. J. Am. Soc. Nephrol. 16:3273-3280 (2005), Gursel, I., et al. (J. Immunol., 171: 1393-1400 (2003), Shirota, H., et al., J. Immunol., 173: 5002-5007 (2004), Chen, Y., et al., Gene Ther. 8: 1024-1032 (2001); Stunz, L. L., Eur. J. Immunol. 32: 1212-1222 (2002; Kandimalla et al. WO2007/7047396). However, oligonucleotides containing guanosine strings have been shown to form tetraplex structures, act as aptamers and inhibit thrombin activity (Bock L C et al., Nature, 355:564-6, 1992; Padmanabhan, K et al., J Biol Chem., 268(24):17651-4, 1993). Thus, the utility of these inhibitory oligodeoxynucleotide molecules may not be achievable in patients.

As an alternative to interacting with the receptor protein and directly inhibiting receptor activation, some studies have suggested the utility of “knock down” or silencing technologies, for example siRNA, miRNA, ddRNA and eiRNA technologies, for inhibiting the activity of a receptor. These technologies rely upon administration or expression of double stranded RNA (dsRNA). However, RNAi molecules act through a catalytic process, these molecules are recognized as being distinct from other technologies that target RNA molecules and inhibit their translation (see for example: Opalinska and Gewirtz (2002) Nature Reviews 1:503-514). Moreover, siRNA molecules have been recognized to induce non-specific immune stimulation through interaction with TLRs (Kleinman et al., (2008) Nature 452:591-597; De Veer et. al. (2005) Immun. Cell Bio. 83:224-228; Kariko et al. (2004) J. Immunol. 172:6545-6549).

A promising approach to suppressing the activity of TLR7 is the use of oligonucleotide-based antagonists (see Kandimalla et al., WO2007/7047396).

Yet another potential approach to “knock down” expression of TLRs is antisense technology. The history of antisense technology has revealed that while discovery of antisense oligonucleotides that inhibit gene expression is relatively straight forward, the optimization of antisense oligonucleotides that have true potential as clinical candidates is not. Accordingly, if an antisense approach to down-regulating TLR7 is to be successful, there is a need for optimized antisense oligonucleotides that most efficiently achieve this result. Such optimized antisense oligonucleotides could be used alone, or in conjunction with the antagonists of Kandimalla et al., or other therapeutic approaches.

### BRIEF SUMMARY OF THE INVENTION

The present invention is directed to optimized synthetic antisense oligonucleotides that are targeted to a nucleic acid encoding TLR7 and that efficiently inhibit the expression of TLR7 through inhibition of mRNA translation and/or through an RNase H mediated mechanism.

In a first aspect, the invention provides for optimized antisense including those

having SEQ IDNOs: 18, 31, 58, 107, 117, 118, 131, 141, 156, 163, 184, 199, 205 or 207.

In a second aspect, the invention provides a composition comprising at least one optimized antisense oligonucleotide according to the invention and a physiologically acceptable carrier, diluent or excipient.

In a third aspect, the invention provides a method of inhibiting TLR7 expression. In this method, an oligonucleotide or multiple oligonucleotides of the invention are specifically contacted or hybridized with TLR7 mRNA either in vitro or in a cell.

In a fourth aspect, the invention provides methods for inhibiting the expression of TLR7 in a mammal, particularly a human, such methods comprising administering to the mammal a compound or composition according to the invention.

In a fifth aspect, the invention provides a method for inhibiting a TLR7-mediated immune response in a mammal, the method comprising administering to the mammal a TLR7 antisense oligonucleotide according to the invention in a pharmaceutically effective amount.

In a sixth aspect, the invention provides a method for therapeutically treating a mammal having a disease mediated by TLR7, such method comprising administering to the mammal, particularly a human, a TLR7 antisense oligonucleotide of the invention, or a composition thereof, in a pharmaceutically effective amount.

In a seventh aspect, the invention provides methods for preventing a disease or disorder in a mammal, particularly a human, at risk of contracting or developing a disease or disorder mediated by TLR7. The method according to this aspect of the invention comprises administering to the mammal an antisense oligonucleotide according to the invention, or a composition thereof, in a prophylactically effective amount.

In an eighth aspect, the invention provides methods for down-regulating TLR7 expression and thus preventing the “off-target” activity of certain other RNA-based molecules, or other compounds or drugs that have a side effect of activating TLR7. For example, the TLR7 antisense oligonucleotide according to the invention can be administered in combination with one or more RNA-based molecules, which are not targeted to the same molecule as the antisense oligonucleotides of the invention, and which comprise an immunostimulatory motif that would activate a TLR7-mediated immune response but for the presence of the TLR7 antisense oligonucleotide according to the invention.

In a ninth aspect, the invention provides a method for inhibiting TLR7 expression and activity in a mammal, comprising administering to the mammal an antisense oligonucleotide complementary to TLR7 mRNA and an antagonist of TLR7 protein, a kinase inhibitor or an inhibitor of STAT (signal transduction and transcription) protein.

The subject oligonucleotides and methods of the invention are also useful for examining the function of the TLR7 gene in a cell or in a control mammal or in a mammal afflicted with a disease associated with TLR7 or immune stimulation through TLR7. The cell or mammal is administered the oligonucleotide, and the expression of TLR7 mRNA or protein is examined.

### BRIEF DESCRIPTION OF THE DRAWINGS

FIG. 1 is a synthetic scheme for the linear synthesis of antisense oligonucleotides of the invention. DMTr=4,4′-dimethoxytrityl; CE=cyanoethyl.

FIG. 2 is a graphical representation of the activity of exemplar human TLR7 antisense oligonucleotides according to the invention in HEK293XL cells expressing human TLR7. The data demonstrate the ability of exemplar oligonucleotides according to the invention to inhibit TLR7 expression and activation in HEK293 cells that were cultured and treated according to Example 2.

FIG. 3 is a graphical representation of the activity of exemplar human TLR7 antisense oligonucleotides according to the invention in HEK293XL cells expressing human TLR7. The data demonstrate the ability of exemplar oligonucleotides according to the invention to inhibit TLR7 expression and activation in HEK293 cells that were cultured and treated according to Example 2.

FIG. 4 is a graphical representation of the activity of exemplar TLR7 antisense oligonucleotides according to the invention to inhibit TLR7-induced IL-12 following in vivo administration according to example 3. The data demonstrate that administration of an exemplar TLR7 antisense oligonucleotide according to the invention can cause down-regulation of TLR7 expression in vivo and prevent the induction of IL-12 by a TLR7 agonist. More generally, the data demonstrate the ability of a TLR7 antisense oligonucleotide according to the invention to inhibit the induction of pro-inflammatory cytokines by a TLR7 agonist.

FIG. 5 depicts human TLR7 mRNA (SEQ ID NO: 258) (GenBank Accession No. AF240467; NM 016562).

### DETAILED DESCRIPTION OF THE PREFERRED EMBODIMENTS

The invention relates to optimized TLR7 antisense oligonucleotides, compositions comprising such oligonucleotides and methods of their use for inhibiting or suppressing a TLR7-mediated response. The antisense oligonucleotides according to the invention are stable, specific and do not activate an innate immune response, thereby overcoming the problems of certain previously attempted approaches. Pharmaceutical and other compositions comprising the compounds according to the invention are also provided. Further provided are methods of down-regulating the expression of TLR7 in cells or tissues comprising contacting said cells or tissues with one or more of the antisense compounds or compositions of the invention alone or in combination with other prophylactic or therapeutic compositions.

Specifically, the invention provides antisense oligonucleotides designed to be complementary to a genomic region or an RNA molecule transcribed therefrom. These TLR7 antisense oligonucleotides have unique sequences that target specific, particularly available mRNA sequences, resulting in maximally effective inhibition or suppression of TLR7-mediated signaling in response to endogenous and/or exogenous TLR7 ligands or TLR7 agonists.

The TLR7 antisense oligonucleotides according to the invention inhibit immune responses induced by natural or artificial TLR7 agonists in various cell types and in various in vitro and in vivo experimental models. As such, the antisense compositions according to the invention are useful as tools to study the immune system, as well as to compare the immune systems of various animal species, such as humans and mice.

Further provided are methods of treating a mammal, particularly a human, having, suspected of having, or being prone to develop a disease or condition associated with TLR7 activation by administering a therapeutically or prophylactically effective amount of one or more of the antisense compounds or compositions of the invention. These can be used for immunotherapy applications such as, but not limited to, treatment of cancer, autoimmune disorders, asthma, respiratory allergies, food allergies, skin allergies, systemic lupus erythematosus (SLE), arthritis, pleurisy, chronic infections, inflammatory diseases, inflammatory bowel syndrome, sepsis, malaria, and bacteria, parasitic, and viral infections in adult and pediatric human and veterinary applications. In addition, The TLR7 antisense oligonucleotides according to the invention are also useful in the prevention and/or treatment of various diseases, either alone, in combination with or co-administered with other drugs or prophylactic or therapeutic compositions, for example, DNA vaccines, antigens, antibodies, and allergens; and in combination with chemotherapeutic agents (both traditional chemotherapy and modern targeted therapies) and/or TLR7 antagonists for prevention and treatment of diseases. TLR7 antisense oligonucleotides of the invention are useful in combination with compounds or drugs that have unwanted TLR7-mediated immune stimulatory properties.

The patents and publications cited herein reflect the level of knowledge in the art and are hereby incorporated by reference in their entirety. Any conflict between the teachings of these patents and publications and this specification shall be resolved in favor of the latter.

The foregoing and other objects of the present invention, the various features thereof, as well as the invention itself may be more fully understood from the following description, when read together with the accompanying drawings in which:

The term “2′-O-substituted” means substitution of the 2′ position of the pentose moiety with an —O— lower alkyl group containing 1-6 saturated or unsaturated carbon atoms (for example, but not limited to, 2′-O-methyl), or with an —O-aryl or allyl group having 2-6 carbon atoms, wherein such alkyl, aryl or allyl group may be unsubstituted or may be substituted, (for example, with 2′-O-ethoxy-methyl, halo, hydroxy, trifluoromethyl, cyano, nitro, acyl, acyloxy, alkoxy, carboxyl, carbalkoxyl, or amino groups); or with a hydroxy, an amino or a halo group, but not with a 2′-H group. In some embodiments the oligonucleotides of the invention include four or five ribonucleotides 2′-O-alkylated at their 5′ terminus (i.e., 5′ 2-O-alkylated ribonucleotides), and/or four or five ribonucleotides 2′-O-alkylated at their 3′ terminus (i.e., 3′ 2-O-alkylated ribonucleotides). In exemplar embodiments, the nucleotides of the synthetic oligonucleotides are linked by at least one phosphorothioate internucleotide linkage. The phosphorothioate linkages may be mixed Rp and Sp enantiomers, or they may be stereoregular or substantially stereoregular in either Rp or Sp form (see Iyer et al. (1995) Tetrahedron Asymmetry 6:1051-1054).

The term “3′”, when used directionally, generally refers to a region or position in a polynucleotide or oligonucleotide 3′ (toward the 3′end of the nucleotide) from another region or position in the same polynucleotide or oligonucleotide.

The term “5′”, when used directionally, generally refers to a region or position in a polynucleotide or oligonucleotide 5′ (toward the 5′ end of the nucleotide) from another region or position in the same polynucleotide or oligonucleotide.

The term “about” generally means that the exact number is not critical. Thus, oligonucleotides having one or two fewer nucleoside residues, or from one to several additional nucleoside residues are contemplated as equivalents of each of the embodiments described above.

The term “agonist” generally refers to a substance that binds to a receptor of a cell and induces a response. An agonist often mimics the action of a naturally occurring substance such as a ligand.

The term “antagonist” generally refers to a substance that attenuates the effects of an agonist.

The term “kinase inhibitor” generally refers to molecules that antagonize or inhibit phosphorylation-dependent cell signaling and/or growth pathways in a cell. Kinase inhibitors may be naturally occurring or synthetic and include small molecules that have the potential to be administered as oral therapeutics. Kinase inhibitors have the ability to rapidly and specifically inhibit the activation of the target kinase molecules. Protein kinases are attractive drug targets, in part because they regulate a wide variety of signaling and growth pathways and include many different proteins. As such, they have great potential in the treatment of diseases involving kinase signaling, including cancer, cardiovascular disease, inflammatory disorders, diabetes, macular degeneration and neurological disorders. Examples of kinase inhibitors include sorafenib (Nexavar®), Sutent®, dasatinib, Dasatinib™, Zactima™, Tykerb™ and STI571.

The term “airway inflammation” generally includes, without limitation, inflammation in the respiratory tract caused by allergens, including asthma.

The term “allergen” generally refers to an antigen or antigenic portion of a molecule, usually a protein, which elicits an allergic response upon exposure to a subject. Typically the subject is allergic to the allergen as indicated, for instance, by the wheal and flare test or any method known in the art. A molecule is said to be an allergen even if only a small subset of subjects exhibit an allergic (e.g., IgE) immune response upon exposure to the molecule.

The term “allergy” generally includes, without limitation, food allergies, respiratory allergies and skin allergies.

The term “antigen” generally refers to a substance that is recognized and selectively bound by an antibody or by a T cell antigen receptor. Antigens may include but are not limited to peptides, proteins, nucleosides, nucleotides and combinations thereof. Antigens may be natural or synthetic and generally induce an immune response that is specific for that antigen.

The term “autoimmune disorder” generally refers to disorders in which “self” antigen undergo attack by the immune system. Such term includes, without limitation, lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis autoimmune asthma, septic shock and psoriasis.

The term “cancer” generally refers to, without limitation, any malignant growth or tumor caused by abnormal or uncontrolled cell proliferation and/or division. Cancers may occur in humans and/or mammals and may arise in any and all tissues. Treating a patient having cancer may include administration of a compound, pharmaceutical formulation or vaccine according to the invention such that the abnormal or uncontrolled cell proliferation and/or division, or metastasis is affected.

The term “carrier” generally encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, for example, Remington's Pharmaceutical Sciences, 18th Edition, ed. A. Gennaro, Mack Publishing Co., Easton, Pa., 1990.

The term “co-administration” or “co-administered” generally refers to the administration of at least two different substances sufficiently close in time to modulate an immune response. Co-administration refers to simultaneous administration, as well as temporally spaced order of up to several days apart, of at least two different substances in any order, either in a single dose or separate doses.

The term “in combination with” generally means administering a compound according to the invention and another agent useful for treating the disease or condition that does not abolish TLR7 antisense activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “individual” or “subject” or “vertebrate” generally refers to a mammal, such as a human.

The term “linear synthesis” generally refers to a synthesis that starts at one end of an oligonucleotide and progresses linearly to the other end. Linear synthesis permits incorporation of either identical or non-identical (in terms of length, base composition and/or chemical modifications incorporated) monomeric units into an oligonucleotide.

The term “mammal” is expressly intended to include warm blooded, vertebrate animals, including, without limitation, humans, non-human primates, rats, mice, cats, dogs, horses, cattle, cows, pigs, sheep and rabbits.

The term “nucleoside” generally refers to compounds consisting of a sugar, usually ribose or deoxyribose, and a purine or pyrimidine base.

The term “nucleotide” generally refers to a nucleoside comprising a phosphorous-containing group attached to the sugar.

The term “modified nucleoside” generally is a nucleoside that includes a modified heterocyclic base, a modified sugar moiety, or any combination thereof. In some embodiments, the modified nucleoside is a non-natural pyrimidine or purine nucleoside, as herein described. For purposes of the invention, a modified nucleoside, a pyrimidine or purine analog or non-naturally occurring pyrimidine or purine can be used interchangeably and refers to a nucleoside that includes a non-naturally occurring base and/or non-naturally occurring sugar moiety. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “modified oligonucleotide” as used herein describes an oligonucleotide in which at least two of its nucleotides are covalently linked via a synthetic linkage, i.e., a linkage other than a phosphodiester linkage between the 5′ end of one nucleotide and the 3′ end of another nucleotide in which the 5′ nucleotide phosphate has been replaced with any number of chemical groups. The term “modified oligonucleotide” also encompasses oligonucleotides having at least one nucleotide with a modified base and/or sugar, such as a 2′-O-substituted, a 5′-O-substituted and/or a 3′-O-substituted ribonucleotide.

The term “nucleic acid” encompasses a genomic region or an RNA molecule transcribed therefrom. In some embodiments, the nucleic acid is mRNA.

The term “nucleotidic linkage” generally refers to a chemical linkage to join two nucleosides through their sugars (e.g. 3′-3′,2′-3′,2′-5′,3′-5′) consisting of a phosphorous atom and a charged, or neutral group (e.g., phosphodiester, phosphorothioate, phosphorodithioate or methylphosphonate) between adjacent nucleosides.

The term “oligonucleotide” refers to a polynucleoside formed from a plurality of linked nucleoside units. The nucleoside units may be part of viruses, bacteria, cell debris or oligonucleotide-based compositions (for example, siRNA and microRNA). Such oligonucleotides can also be obtained from existing nucleic acid sources, including genomic or cDNA, but are preferably produced by synthetic methods. In certain embodiments each nucleoside unit includes a heterocyclic base and a pentofuranosyl, trehalose, arabinose, 2′-deoxy-2′-substituted nucleoside, 2′-deoxy-2′-substituted arabinose, 2′-O-substitutedarabinose or hexose sugar group. The nucleoside residues can be coupled to each other by any of the numerous known internucleoside linkages. Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide-based compound” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain exemplar embodiments, these internucleoside linkages may be phosphodiester, phosphorothioate or phosphorodithioate linkages, or combinations thereof.

The term “complementary to a genomic region or an RNA molecule transcribed therefrom” is intended to mean an oligonucleotide that binds to the nucleic acid sequence under physiological conditions, for example, by Watson-Crick base pairing (interaction between oligonucleotide and single-stranded nucleic acid) or by Hoogsteen base pairing (interaction between oligonucleotide and double-stranded nucleic acid) or by any other means, including in the case of an oligonucleotide, binding to RNA and causing pseudoknot formation. Binding by Watson-Crick or Hoogsteen base pairing under physiological conditions is measured as a practical matter by observing interference with the function of the nucleic acid sequence.

The term “peptide” generally refers to polypeptides that are of sufficient length and composition to affect a biological response, for example, antibody production or cytokine activity whether or not the peptide is a hapten. The term “peptide” may include modified amino acids (whether or not naturally or non-naturally occurring), where such modifications include, but are not limited to, phosphorylation, glycosylation, pegylation, lipidization and methylation.

The term “pharmaceutically acceptable” means a non-toxic material that does not interfere with the effectiveness of a compound according to the invention or the biological activity of a compound according to the invention.

The term “physiologically acceptable” refers to a non-toxic material that is compatible with a biological system such as a cell, cell culture, tissue, or organism. Preferably, the biological system is a living organism, such as a mammal, particularly a human.

The term “prophylactically effective amount” generally refers to an amount sufficient to prevent or reduce the development of an undesired biological effect.

The term “therapeutically effective amount” or “pharmaceutically effective amount” generally refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful patient benefit, for example, but not limited to, healing of chronic conditions characterized by immune stimulation. Thus, a “pharmaceutically effective amount” will depend upon the context in which it is being administered. A pharmaceutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “treatment” generally refers to an approach intended to obtain a beneficial or desired result, which may include alleviation of symptoms, or delaying or ameliorating a disease progression.

In a first aspect, the invention provides antisense oligonucleotides that are complementary to a nucleic acid that is specific for human TLR7 (SEQ ID NO: 258). The antisense oligonucleotides according to the invention are optimized with respect to the targeted region of the TLR7 mRNA coding sequence, or 5′ untranslated region or the 3′ untranslated region, in their chemical modification, and or both. In some embodiments of this aspect, the compounds are complementary to a region within nucleobases 140 through 3289 of the coding region, or 1-139 of the 5′ untranslated region, or 3290-4192 of the 3′ untranslated region of TLR7 mRNA. (SEQ ID NO: 258).

Antisense oligonucleotides according to the invention are useful in treating and/or preventing diseases wherein inhibiting a TLR7-mediated immune response would be beneficial. TLR7-targeted antisense oligonucleotides according to the invention that are useful include, but are not limited to, antisense oligonucleotides comprising naturally occurring nucleotides, modified nucleotides, modified oligonucleotides and/or backbone modified oligonucleotides. However, antisense oligonucleotides that inhibit the translation of mRNA encoded proteins may produce undesired biological effects, including but not limited to insufficiently active antisense oligonucleotides, inadequate bioavailability, suboptimal pharmacokinetics or pharmacodynamics, and immune stimulation. Thus, the optimal design of an antisense oligonucleotide according to the invention requires many considerations beyond simple design of a complementary sequence. Thus, preparation of TLR7-targeted antisense oligonucleotides according to the invention is intended to incorporate changes necessary to limit secondary structure interference with antisense activity, enhance the oligonucleotide's target specificity, minimize interaction with binding or competing factors (for example, proteins), optimize cellular uptake, stability, bioavailability, pharmacokinetics and pharmacodynamics, and/or inhibit, prevent or suppress immune cell activation. Such inhibition, prevention or suppression of immune cell activation may be accomplished in a number of ways without compromising the antisense oligonucleotide's ability to hybridize to nucleotide sequences contained within the mRNA for TLR7, including, without limitation, incorporation of one or more modified nucleotides or nucleotide linkages, wherein such modified nucleotides are a 2′-O-methyl, a 3′-O-methyl, a 5-methyl, a 2′-O-methoxyethyl-C, a 2′-O-methoxyethyl-5-methyl-C and/or a 2′-O-methyl-5-methyl-C on the “C” of a “CpG” dinucleotide, a 2′-O-substituted-G, a 2′-O-methyl-G and/or a 2′-O-methoxyethoxy-G on the “G” of the CpG, and such modified nucleotide linkages are a non-phosphate or non-phosphorothioate internucleoside linkage between the C and G of a “CpG” dinucleotide, a methylphosphonate linkage and/or a 2′-5′ internucleotide linkage between the C and G of a “CpG” dinucleotide.

It has been determined that the human TLR7 mRNA coding region is comprised of approximately 3.1 kB, and the transcript corresponding to the 1049 amino acid protein has also been identified in humans (Chuang and Ulevitch, Eur. Cytokine Network (2000) 3:372-378). The sequence of the gene encoding TLR7 has been reported in mice (Hemmi et al., Nature (2000) 408:740-745) and for humans (Chuang and Ulevitch, Eur. Cytokine Network (2000) 3:372-378). The oligonucleotides of the invention are directed to optimally available portions of the TLR7 nucleic acid sequence that most effectively act as a target for inhibiting TLR7 expression. These targeted regions of the TLR7 gene include portions of the known exons or 5′ untranslated region. In addition, intron-exon boundaries, 3′ untranslated regions and introns are potentially useful targets for antisense inhibition of TLR7 expression. The nucleotide sequences of some representative, non-limiting oligonucleotides specific for human TLR7 have SEQ ID NOS: 1-257. The nucleotide sequences of optimized oligonucleotides according to the invention include those having SEQ ID NOS: 18, 31, 58, 107, 117, 118, 131, 141, 156, 163, 184, 199, 205 or 207.

The oligonucleotides of the invention are composed of ribonucleotides, deoxyribonucleotides or a combination of both, with the 5′ end of one nucleotide and the 3′ (or in limited cases 2′) end of another nucleotide being covalently linked. The oligonucleotides of the invention are at least 14 nucleotides in length, but are preferably 15 to 60 nucleotides long, preferably 20 to 50 nucleotides in length. In some embodiments, these oligonucleotides contain from about 14 to 28 nucleotides or from about 16 to 25 nucleotides or from about 18 to 22 nucleotides or 20 nucleotides. These oligonucleotides can be prepared by the art recognized methods such as phosphoramidate or H-phosphonate chemistry which can be carried out manually or by an automated synthesizer. The synthetic TLR7 antisense oligonucleotides of the invention may also be modified in a number of ways without compromising their ability to hybridize to TLR7 mRNA. Such modifications may include at least one internucleotide linkage of the oligonucleotide being an alkylphosphonate, phosphorothioate, phosphorodithioate, methy phosphonate, phosphate ester, alkylphosphonothioate, phosphoramidate, carbamate, carbonate, phosphate triester, acetamidate or carboxymethyl ester or a combination of these and other internucleotide linkages between the 5′ end of one nucleotide and the 3′ end of another nucleotide in which the 5′ nucleotide phosphodiester linkage has been replaced with any number of chemical groups.

For example, U.S. Pat. No. 5,149,797 describes traditional chimeric oligonucleotides having a phosphorothioate core region interposed between methylphosphonate or phosphoramidate flanking regions. U.S. Pat. No. 5,652,356 discloses “inverted” chimeric oligonucleotides comprising one or more nonionic oligonucleotide region (e.g. alkylphosphonate and/or phosphoramidate and/or phosphotriester internucleoside linkage) flanked by one or more region of oligonucleotide phosphorothioate. Various oligonucleotides with modified internucleotide linkages can be prepared according to standard methods. Phosphorothioate linkages may be mixed Rp and Sp enantiomers, or they may be made stereoregular or substantially stereoregular in either Rp or Sp form according to standard procedures.

Oligonucleotides which are self-stabilized are also considered to be modified oligonucleotides useful in the methods of the invention (Tang et al. (1993) Nucleic Acids Res. 20:2729-2735). These oligonucleotides comprise two regions: a target hybridizing region; and a self-complementary region having an oligonucleotide sequence complementary to a nucleic acid sequence that is within the self-stabilized oligonucleotide.

Other modifications include those which are internal or at the end(s) of the oligonucleotide molecule and include additions to the molecule of the internucleoside phosphate linkages, such as cholesterol, cholesteryl, or diamine compounds with varying numbers of carbon residues between the amino groups and terminal ribose, deoxyribose and phosphate modifications which cleave, or crosslink to the opposite chains or to associated enzymes or other proteins which bind to the genome. Examples of such modified oligonucleotides include oligonucleotides with a modified base and/or sugar such as arabinose instead of ribose, or a 3′, 5′-substituted oligonucleotide having a sugar which, at both its 3′ and 5′ positions, is attached to a chemical group other than a hydroxyl group (at its 3′ position) and other than a phosphate group (at its 5′ position).

Other examples of modifications to sugars include modifications to the 2′ position of the ribose moiety which include but are not limited to 2′-O-substituted with an —O-alkyl group containing 1-6 saturated or unsaturated carbon atoms, or with an —O-aryl, or —O-allyl group having 2-6 carbon atoms wherein such —O-alkyl, —O-aryl or —O-allyl group may be unsubstituted or may be substituted, for example with halo, hydroxy, trifluoromethyl cyano, nitro acyl acyloxy, alkoxy, carboxy, carbalkoxyl or amino groups. None of these substitutions are intended to exclude the native 2′-hydroxyl group in the case of ribose or 2′1-H— in the case of deoxyribose.

U.S. Pat. No. 5,652,355 discloses traditional hybrid oligonucleotides having regions of 2′-O-substituted ribonucleotides flanking a DNA core region. U.S. Pat. No. 5,652,356 discloses an “inverted” hybrid oligonucleotide which includes an oligonucleotide comprising a 2′-O-substituted (or 2′ OH, unsubstituted) RNA region which is in between two oligodeoxyribonucleotide regions, a structure that “inverted relative to the “traditional” hybrid oligonucleotides. Non-limiting examples of particularly useful oligonucleotides of the invention have 2′-O-alkylated ribonucleotides at their 3′, 5′, or 3′ and 5′ termini, with at least four or five contiguous nucleotides being so modified. Non-limiting examples of 2′-O-alkylated groups include 2′-O-methyl, 2′-O-ethyl, 2′-O-propyl, 2′-O-butyls and 2′-O-ethoxy-methyl.

Other modified oligonucleotides are capped with a nuclease resistance-conferring bulky substituent at their 3′ and/or 5′ end(s), or have a substitution in one non-bridging oxygen per nucleotide. Such modifications can be at some or all of the internucleoside linkages, as well as at either or both ends of the oligonucleotide and/or in the interior of the molecule.

The oligonucleotides of the invention can be administered in combination with one or more antisense oligonucleotides or other nucleic acid containing compounds, which are not the same target as the antisense molecule of the invention, and which comprise an immunostimulatory motif that would activate a TLR7-mediated immune response but for the presence of the TLR7 antisense oligonucleotide according to the invention. In addition, the oligonucleotides of the invention can be administered in combination with one or more vaccines, antigens, antibodies, cytotoxic agents, allergens, antibiotics, TLR antagonists, siRNA, miRNA, antisense oligonucleotides, aptamers, peptides, proteins, gene therapy vectors, DNA vaccines, adjuvants, kinase inhibitors or co-stimulatory molecules or combinations thereof.

A non-limiting list of TLR7 antisense oligonucleotides are shown in SEQ ID NO. 1 through SEQ ID NO 257 and Table 2 below. Optimized antisense oligonucleotides according to the invention include those having SEQ ID NOS: 18, 31, 58, 107, 117, 118, 131, 141, 156, 163, 184, 199, 205 or 207. In Table 2, the oligonucleotide-based TLR7 antisense compounds have all phosphorothioate (PS) linkages. Those skilled in the art will recognize, however, that phosphodiester (PO) linkages, or a mixture of PS and PO linkages can be used.

TABLE 2

| SEQ | | |
|---|---|---|
| ID | Position | Antisense Sequence |
| NO. | of Binding | Orientation is 5′-3′ |
| 1 | 1 | ATCCTATATC TGGAGTCTTC |
| 2 | 17 | TTGATGGCAT GGAGTGATCC |
| 3 | 21 | TTTCTTGATG GCATGGAGTG |
| 4 | 41 | ATGGGCCCAA TAGCATCAAC |
| 5 | 61 | GGTGCCAAGA TCAGCTTGAG |
| 6 | 75 | AGCAGAGCAT GAGAGGTGCC |
| 7 | 81 | GAAGAGAGCA GAGCATGAGA |
| 8 | 101 | TGGAATGTAG AGGTCTGGTT |
| 9 | 121 | TTTTTAGTCT TCTTCCAAAA |
| 10 | 141 | TGTCCACATT GGAAACACCA |
| 11 | 154 | TTTGTCTCTT CAGTGTCCAC |
| 12 | 161 | ATAAGAATTT GTCTCTTCAG |
| 13 | 181 | TTAGGATTAT GTTAAAAAGG |
| 14 | 201 | AGCCCCAAGG AGTTTGGAAA |
| 15 | 221 | AGAGTTTTAG GAAACCATCT |
| 16 | 241 | CCAGAGTGAC ATCACAGGGC |
| 17 | 261 | CACATGGTTC TTTGGAACAT |
| 18 | 284 | TGCTTGTCTG TGCAGTCCAC |
| 19 | 301 | CAGGAATTTC TGTCAAATGC |
| 20 | 321 | GGTGTTCGTG GGAATACCTC |
| 21 | 341 | ATGGTGAGGG TGAGGTTCGT |
| 22 | 361 | AGATGTCTGG TATGTGGTTA |
| 23 | 381 | TCTGTGAAAG GACGCTGGGG |
| 24 | 401 | ATCTCTACCA GATGGTCCAG |
| 25 | 421 | CACAGTTGCA TCTGAAATCG |
| 26 | 441 | TGACCCCAGT GGAATAGGTA |
| 27 | 461 | TTGATGCACA TGTTGTTTTT |
| 28 | 481 | TGGGTTTAAT CTGCAGCCTC |
| 29 | 501 | AGTGAGTCCA CTAAAGCTTC |
| 30 | 521 | AGGTAAAGGG ATTTTAAATA |
| 31 | 541 | CTAGTAGCTG GTTTCCATCC |
| 32 | 561 | CGGGAGGCCC TGCGGTATCT |
| 33 | 581 | CTGAGAAGCT GTAAGCTAGG |
| 34 | 601 | AGATGTTGTT GGCCTCAAGG |
| 35 | 621 | ATTCTCTTTT CTGATGGAAA |
| 36 | 641 | ATGTTGGCCA GTTCTGTTAG |
| 37 | 661 | GGCCCAGGTA GAGTATTTCT |
| 38 | 681 | ATTTCGATAA TAACAGTTTT |
| 39 | 701 | GAATATGAAA CATAACAAGG |
| 40 | 721 | GGAAGGCATC TTTCTCTATT |
| 41 | 741 | TTTTAACTTT GTCAAGTTTA |
| 42 | 761 | TTATCTTTCA GGGAGAGCAC |
| 43 | 781 | TAGGGACGGC TGTGACATTG |
| 44 | 801 | TAAAGTAGAT GGCAAAACAG |
| 45 | 821 | TTGTAGAGAT ATAGTTCTGT |
| 46 | 841 | GGATTTTTGC AATCATGTTG |
| 47 | 861 | GTTATTAAAA TCATCTTCTT |
| 48 | 881 | AGAATTTGTA ATTGGTTGAG |
| 49 | 901 | GGCAATTTCC ACTTAGGTCA |
| 50 | 921 | TGGGGCATTA TAACAACGAG |
| 51 | 941 | TTACACGGCG CACAAGGAAA |
| 52 | 961 | TCTGTAGGGG AGAATTATTT |
| 53 | 981 | ATCAAAAGCA TTTACAGGGA |
| 54 | 1001 | ACTTTTAATT CTGTCAGCGC |
| 55 | 1021 | AGTTACTGTG TAGACGTAAA |
| 56 | 1041 | TGGGGGCACA TGCTGAAGAG |
| 57 | 1061 | TTGATGTTCT TAAACCATCT |
| 58 | 1072 | CCTGGAGTTT GTTGATGTTC |
| 59 | 1081 | GATCCAGTTC CTGGAGTTTG |
| 60 | 1101 | GGCCAAGAAG TTTTGGGACA |
| 61 | 1121 | TTAGCATCCC CAATTTCTTT |
| 62 | 1141 | TGGGGAGAAA ATGCAGAAAT |
| 63 | 1161 | CAGATCCAAT TGGATGAGGC |
| 64 | 1181 | TGAAGTTCAA AATTGAAAGA |
| 65 | 1201 | TCATAGATGC ACGATAGACC |
| 66 | 1221 | AGAAAATGCT TGTGATAGAT |
| 67 | 1241 | ATTTTCAGGC TTTTCAGTGA |
| 68 | 1261 | CATATCCTCT GATCCGCAGA |
| 69 | 1281 | GCTTTTCAAC TCTTTAAAGA |
| 70 | 1301 | TGTAATGGCG AGAGGTTAAA |
| 71 | 1321 | CTTCAAGATT TTGAAGATTA |
| 72 | 1341 | GTTAGTGCCA AGATCAAGAA |
| 73 | 1361 | AGGTTAGCAA TTTTTATAAA |
| 74 | 1381 | TAAATTGTTT AAACATGCTG |
| 75 | 1401 | ATCTATGACT TTCAGTCTTT |
| 76 | 1421 | GATATTTTAT TCACTGAAAG |
| 77 | 1441 | CACTTGAATC TCCTGAAGGT |
| 78 | 1467 | TCTGGCATTT GAGCAGAAGC |
| 79 | 1481 | CTTTCTACAG AAGTTCTGGC |
| 80 | 1501 | CCAGGACCTG GGGTTCATAA |
| 81 | 1521 | TCTGAAATAA TGTAATTGTT |
| 82 | 1541 | CTCCTTGCAT ACTTATCATA |
| 83 | 1561 | CTTTGTTTTT GAATCTGCAA |
| 84 | 1581 | AACAGACATG AAAGAAGCCT |
| 85 | 1601 | TACTTGTAGC AGCTTTCATT |
| 86 | 1621 | TTAGATCCAA GGTCTGCCCA |
| 87 | 1641 | AAAAAATATA CTATTTTTAC |
| 88 | 1661 | TGAAAATCAG AGGACTTGAC |
| 89 | 1681 | ATTTGAGGAA AGAAAGATGC |
| 90 | 1701 | ATTTCCTGAC AGATTCAGGC |
| 91 | 1721 | TTAAGAGTTT GGCTAATGAG |
| 92 | 1741 | AAGGTTGGAA TTCACTGCCA |
| 93 | 1761 | CAAATATCTC AGCTCTGCTA |
| 94 | 1781 | AGCCGGTTGT TGGAGAAGTC |
| 95 | 1801 | CTGTTGAATG GAGTAAATCA |
| 96 | 1821 | TTTGTGAAGC TCTTCAAATG |
| 97 | 1841 | CTTATATCCA GAACTTCCAG |
| 98 | 1861 | GAAAATAATG GCTATTACTG |
| 99 | 1881 | ATGAGTAATT CCTTCTGATT |
| 100 | 1901 | TTCTTGGTAA AGTTTAGCAT |
| 101 | 1921 | GTTTCTGCAG AACCTTTAGG |
| 102 | 1941 | GTCATTGTCG TTCATCATCA |
| 103 | 1961 | CTGCTGGTGG AGGAAGAGAT |
| 104 | 1981 | GAGACTCACT CTCCATGGTC |
| 105 | 2001 | TCTGAATTCC AGAGTTCTAA |
| 106 | 2021 | AAAACATCTA AGTGATTTCC |
| 107 | 2043 | TCTGTTATCA CCTTCTCTCC |
| 108 | 2061 | CTTGAATAAT TGTAAGTATC |
| 109 | 2081 | TCCTCTAATT TTAGCAGATT |
| 110 | 2101 | AATTTTTAGA GATGTCTAAT |
| 111 | 2121 | AGAAGGCAAG AAACTTAGGG |
| 112 | 2141 | GGCATACCAT CAAAAACTCC |
| 113 | 2161 | AGAGATTCTT TAGATTTGGA |
| 114 | 2181 | GAGCCCATTT TTGGCCAAAG |
| 115 | 2201 | TTCTTCCAAC TGAAAGATTT |
| 116 | 2221 | GGTTCTTTAG ACACTGGAGT |
| 117 | 2236 | GGTCCAAAGT TTCCAGGTTC |
| 118 | 2251 | GTTGGTTGTG GCTGAGGTCC |
| 119 | 2261 | ACAGTGGTCA GTTGGTTGTG |
| 120 | 2281 | AGTTGGATAA TCTCTCAGGG |
| 121 | 2301 | ATTCTTGAGG CTTCTGGAAC |
| 122 | 2321 | TGATTATTCT TAAGAATCAG |
| 123 | 2341 | ACTTCGTCAG ACTCCTGATT |
| 124 | 2361 | GAAGGCATCT TGTAGAAAAT |
| 125 | 2381 | AGATCCAGAT ATCGCAACTG |
| 126 | 2401 | TCTGGATTTT ATTTGAGCTG |
| 127 | 2421 | GAAGCTGGTC TTTTGGATCA |
| 128 | 2441 | TTGTTGAGGA CATTTTCTGG |
| 129 | 2461 | GCAAAAGCAA CATCTTCAGA |
| 130 | 2481 | GCACAGAAAC CGATTATGAT |
| 131 | 2499 | CCACACAGCA TCACAGGTGC |
| 132 | 2521 | TATGGTTAAC CCACCAGACA |
| 133 | 2541 | GTAAGGAATA GTCACCTCCG |
| 134 | 2561 | CAAGTCACAT CTGTGGCCAG |
| 135 | 2581 | TGTGTGCTCC TGGCCCCACA |
| 136 | 2601 | GGAGATCACA CTTTGGCCCT |
| 137 | 2621 | TCACAGGTGT ACAGATCCAG |
| 138 | 2641 | TCAGGTTAGT CAGATCTAAC |
| 139 | 2661 | TATGGAAAGT GAGAACAGAA |
| 140 | 2681 | ATGAGAAAGA GAGATACAGA |
| 141 | 2701 | GACTTGCTGT CATCATCACC |
| 142 | 2721 | CACATCCCAG AAATAGAGGT |
| 143 | 2741 | CAGAAATGGT AAATATACCA |
| 144 | 2761 | ACCCCTTTA CTTGGCCTTA |
| 145 | 2781 | TGGTGATATT AGACGCTGAT |
| 146 | 2801 | AAAGCATCAT AGCAACAGTC |
| 147 | 2821 | CTTTAGTGTC ATACACAATA |
| 148 | 2841 | CCACTCGGTC ACAGCTGGGT |
| 149 | 2861 | GCCACCAGCT CAGCCAAAAC |
| 150 | 2881 | CTCTTGGGTC TTCCAGTTTG |
| 151 | 2901 | ACATAAATTA AAATGTTTCT |
| 152 | 2921 | AACCAGTCCC TTTCCTCGAG |
| 153 | 2941 | CCAGAACTGG CTGCCCTGGT |
| 154 | 2961 | TATGCTCTGG GAAAGGTTTT |
| 155 | 2981 | ACTGTCTTTT TGCTAAGCTG |
| 156 | 3005 | GCATACTTGT CTGTCATCAC |
| 157 | 3021 | AAAATTTTCA GTCTTTGCAT |
| 158 | 3041 | GACAAGTAAA ATGCTATCTT |
| 159 | 3061 | CATCCATGAG CCTCTGATGG |
| 160 | 3081 | GATAATCACA TCAACTTTTT |
| 161 | 3101 | GGCTTCTCAA GAAATATCAA |
| 162 | 3121 | GGAACTTGGA CTTCTGAAAG |
| 163 | 3127 | GCTGGAGGAA CTTGGACTTC |
| 164 | 3141 | GAGCCTTTTC CGGAGCTGGA |
| 165 | 3161 | TCAAGGACAG AACTCCCACA |
| 166 | 3181 | CTTGCGGGTT TGTTGGCCAC |
| 167 | 3201 | CTGCCAGAAG TATGGGTGAG |
| 168 | 3221 | GCCAGGGCGT TCTTTAGACA |
| 169 | 3241 | AGGCCACATG ATTGTCTGTG |
| 170 | 3261 | TTCCTTGAAC ACCTGACTAT |
| 171 | 3281 | GCAAAGAAGG GCTAGACCGT |
| 172 | 3301 | AAACTAGGCA GTTGTGTTTT |
| 173 | 3321 | CAGCCAGGCC TCTCCTTGGT |
| 174 | 3341 | ATATATGAAA ACAATTTAAA |
| 175 | 3361 | AAACACGCTT TTGGTGTGAT |
| 176 | 3381 | TCATTTCTTG AAGAATTTCA |
| 177 | 3401 | CCCCTGAAAT ATGGGCAATC |
| 178 | 3421 | TGTGACAGAC GTTGGTGGCT |
| 179 | 3441 | AACCCCATCT TTCCAACTCC |
| 180 | 3461 | AAGACTTGAT GCATTATATA |
| 181 | 3481 | GACACAGAGA GATAAGAAAG |
| 182 | 3501 | GAGACTCAAG TGCAAATAGA |
| 183 | 3521 | TTTTACAGGA GCTGAGGTGA |
| 184 | 3529 | TGCCACTCTT TTACAGGAGC |
| 185 | 3541 | ATGTTTTTTA CTTGCCACTC |
| 186 | 3561 | TACAGGAGAA TCAGAGCCCC |
| 187 | 3581 | GTATATTTAA TTATCACAAT |
| 188 | 3601 | TCTCAATGTC ATGATTGTGT |
| 189 | 3621 | AAGGGTAGAA ATGCAGTTCT |
| 190 | 3641 | TGTATATACC AGTACTTTTT |
| 191 | 3661 | GTTTTTTTTA ACCCTATTTC |
| 192 | 3681 | CTCATATAGA GAGAGCTTGA |
| 193 | 3701 | TAACTCTAGT ACATTTTGGT |
| 194 | 3721 | GGTTTTTTAT TTCACTAAAC |
| 195 | 3741 | CCATGCCCGG CCAGCTGACT |
| 196 | 3761 | GGGATTACAA GCATGAGCCA |
| 197 | 3781 | CCTCGGCCTC CCAAAGTGCT |
| 198 | 3801 | CTGACCTCGT GATCCACCTG |
| 199 | 3816 | ACTGGTCTCA AACTCCTGAC |
| 200 | 3820 | CCAGACTGGT CTCAAACTCC |
| 201 | 3841 | GACGGGGTTT CACCATGTTG |
| 202 | 3861 | ATTTTTGTAT TTTTAGTACA |
| 203 | 3881 | CCCACCACCA CGCCCAGCTA |
| 204 | 3901 | AGTAGCTGGG ATTACAGGCA |
| 205 | 3924 | GATTCTCCTG CCTCAGCCTC |
| 206 | 3941 | ACCTCCCGGG TTCAAGCGAT |
| 207 | 3958 | GGCTCACTGC CACCTCCACC |
| 208 | 3981 | CTGCATTGCA GTGGCGTGAT |
| 209 | 4001 | GTCTAGCTCT GTTGCCCGGG |
| 210 | 4021 | TTTTTTGTTC TTTTGAGACA |
| 211 | 4041 | GAGTTTTTTT GTGTTTTTTT |
| 212 | 4061 | AATTGGTTAA GAAGCTGACT |
| 213 | 4081 | GCCCTGGATG ACACGGAAGC |
| 214 | 4101 | CTCAATCTGC ACAGAATGGG |
| 215 | 4121 | CCACCTGTGT GGTGCCCACA |
| 216 | 4141 | AGGAAGCACT GAAGCAGCAA |
| 217 | 4161 | CAGGCCCAAG GAAAAAGAGC |
| 218 | 4181 | TTCCCTATGG AACCCAGAAG |
| 219 | 4201 | GTGTCTTTCT TTCTTACTGT |
| 220 | 4221 | TATGCATTTA TGGTAAGGAT |
| 221 | 4241 | TTCTATTTGT AGGTGGACCA |
| 222 | 4261 | GGCAGATCAT TTAAATATTT |
| 223 | 4281 | AGAATATCAC TTTGTATAAA |
| 224 | 4301 | AGGTAAATTA TCAAAGGTAG |
| 225 | 4321 | GCAGATAAAA ACATTTAAGC |
| 226 | 4341 | TGGATACAGT ACTTTGCAGT |
| 227 | 4361 | TGGATGAGGA AATTTTACTT |
| 228 | 4381 | CAAAACAGTT TGAAAGATAT |
| 229 | 4401 | AAATATATGG CATTAGTTAA |
| 230 | 4421 | TCAAGTGTGC AGATACTTAC |
| 231 | 4441 | AACCATCTAA CGTTGCTGTA |
| 232 | 4461 | CCTTTAGGGT TTACCATCAA |
| 233 | 4481 | ATACACACTC TTGGAGTCCT |
| 234 | 4501 | TCTGATAAAA CTATAAATAA |
| 235 | 4521 | GCATTCAAAT AATTGTCATC |
| 236 | 4541 | GAAAGGAATC CATATAATTG |
| 237 | 4561 | CCCATCCTCC AGCAAAAAAT |
| 238 | 4581 | TATAAACTTT GGTTTCTTCT |
| 239 | 4601 | GCTTTCTCAA TGTGAAGGTC |
| 240 | 4621 | AGCTGAAGTT CAAAACTGAA |
| 241 | 4641 | CTGTTGTTTT TGAATCTGAT |
| 242 | 4661 | TAAGAATGTC TTGGTTCTTT |
| 243 | 4681 | AGCTGAAAGT ACAGGCATCT |
| 244 | 4701 | GAACTCATGA ATTTATACCC |
| 245 | 4721 | TTGGTCAGGT TTCAATCTTT |
| 246 | 4741 | CTTCCATGAA ATAAAGCAAA |
| 247 | 4761 | AACACCTTTG TAGATCACTT |
| 248 | 4781 | GCTGTTTTCC AAATGGCACA |
| 249 | 4801 | TAAGGCTTGA ACACATGCAC |
| 250 | 4821 | AAAATACGAC ATCGCCAATC |
| 251 | 4841 | TGGCATTGCC ACACGTGAGG |
| 252 | 4861 | CACAGGTAAA GTAAAGCCTT |
| 253 | 4881 | TAATTCATAT AGTGTGTACT |
| 254 | 4901 | GATTAAATGT ACGTTGGAAA |
| 255 | 4921 | GGGAATTTGT GACCCTTATT |
| 256 | 4941 | TTTATTCCAG AGATTGATTT |
| 257 | 4961 | GCAATTTAAT TACCTCTCTA |
| 259 | 335 (mouse) | AATGCTTGTC TGTGCAGTCC (MOUSE) |
| 260 | 683 (mouse) | CCAGTTCTGT TAGATTCTCC (MOUSE) |
| 261 | 2061 (mouse) | TGGTTGCCTC TGAACTCCAG (MOUSE) |
| 262 | 2634 (mouse) | TTGTGTGCTC CTGGACCTAC (MOUSE) |

Underlined nucleotides are 2′-O-methylribonucleotides; all others are 2′-deoxyribonucleotides. All sequences are phosphorothioate backbone modified. In the exemplar antisense oligonucleotides according to the invention, when a “CG” dinucleotide is contained in the sequence, such oligonucleotide is modified to remove or prevent the immune stimulatory properties of the oligonucleotide.

In a second aspect, the invention provides a composition comprising at least one optimized antisense oligonucleotide according to the invention and a physiologically acceptable carrier, diluent or excipient. The characteristics of the carrier will depend on the route of administration. Such a composition may contain, in addition to the synthetic oligonucleotide and carrier, diluents, fillers, salts, buffers, stabilizers, solubilizers, and other materials well known in the art. The pharmaceutical composition of the invention may also contain other active factors and/or agents which enhance inhibition of TLR7 expression. For example, combinations of synthetic oligonucleotides, each of which is directed to different regions of the TLR7 mRNA, may be used in the pharmaceutical compositions of the invention. The pharmaceutical composition of the-invention may further contain nucleotide analogs such as azidothymidine, dideoxycytidine, dideoxyinosine, and the like. Such additional factors and/or agents may be included in the pharmaceutical composition to produce a synergistic, additive or enhanced effect with the synthetic oligonucleotide of the invention, or to minimize side-effects caused by the synthetic oligonucleotide of the invention. The pharmaceutical composition of the invention may be in the form of a liposome in which the synthetic oligonucleotides of the invention is combined, in addition to other pharmaceutically acceptable carriers, with amphipathic agents such as lipids which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of such liposomal formulations is within the level of skill in the art, as disclosed, for example, in U.S. Pat. Nos. 4,235,871; 4,501,728; 4,837,028; and 4,737,323. The pharmaceutical composition of the invention may further include compounds such as cyclodextrins and the like that enhance delivery of oligonucleotides into cells or slow release polymers.

In a third aspect, the invention provides a method of inhibiting TLR7 expression. In this method, an oligonucleotide or multiple oligonucleotides of the invention are specifically contacted or hybridized with TLR7 mRNA either in vitro or in a cell.

In a fourth aspect, the invention provides methods for inhibiting the expression of TLR7 in an mammal, particularly a human, such methods comprising administering to the mammal a compound or composition according to the invention.

In a fifth aspect, the invention provides a method for inhibiting a TLR-mediated immune response in a mammal, the method comprising administering to the mammal a TLR7 antisense oligonucleotide according to the invention in a pharmaceutically effective amount, wherein routes of administration include, but are not limited to, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

In a sixth aspect, the invention provides a method for therapeutically treating a mammal having a disease mediated by TLR7, such method comprising administering to the mammal, particularly a human, a TLR7 antisense oligonucleotide of the invention in a pharmaceutically effective amount.

In certain embodiments, the disease is cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a disease caused by a pathogen. Preferred autoimmune disorders include without limitation lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. In certain embodiments, inflammatory disorders include without limitation airway inflammation, asthma, autoimmune diseases, chronic inflammation, chronic prostatitis, glomerulonephritis, Behçcet's disease, hypersensitivities, inflammatory bowel disease, reperfusion injury, rheumatoid arthritis, transplant rejection, ulcerative colitis, uveitis, conjunctivitis and vasculitis.

In a seventh aspect, the invention provides methods for preventing a disease or disorder in a mammal, particularly a human, at risk of contracting or developing a disease or disorder mediated by TLR7. The method according to this aspect comprises administering to the mammal a prophylactically effective amount of an antisense oligonucleotide or composition according to the invention. Such diseases and disorders include, without limitation, cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a disease caused by a pathogen in a mammal. Autoimmune disorders include, without limitation, lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. Inflammatory disorders include, without limitation, airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

In an eighth aspect of the invention, the invention provides methods for down-regulating TLR7 expression and thus preventing the “off-target” activity of certain other antisense molecules, or other compounds or drugs that have a side effect of activating TLR7. Certain antisense and other DNA and/or RNA-based compounds that are designed to down-regulate expression of targets other than TLR7 also are recognized by TLR7 proteins and induce an immune response. This activity can be referred to as “off-target” effects. The TLR7 antisense oligonucleotides according to the invention have the ability to down-regulate TLR7 expression and thus prevent the TLR7-mediated off-target activity of the non-TLR7 targeted antisense molecules. For example, the TLR7 antisense oligonucleotide according to the invention can be administered in combination with one or more antisense oligonucleotides, which are not the same target as the antisense molecule of the invention, and which comprise an immunostimulatory motif that would activate a TLR7-mediated immune response but for the presence the TLR7 antisense oligonucleotide according to the invention. Thus, for example, the TLR7 antisense oligonucleotide may be administered in combination with one or more antisense oligonucleotides or RNAi molecules (for example: siRNA, miRNA, ddRNA and eiRNA), which are not targeted to the same molecule as the antisense oligonucleotides of the invention.

In a ninth aspect, the invention provides a method for inhibiting TLR7 expression and activity in a mammal, comprising administering to the mammal an antisense oligonucleotide complementary to TLR7 mRNA and an antagonist of TLR7 protein, a kinase inhibitor or an inhibitor of STAT (signal transduction and transcription) protein. According to this aspect, TLR7 expression is inhibited by the antisense oligonucleotide, while any TLR7 protein residually expressed is inhibited by the antagonist. Preferred antagonists include anti-TLR7 antibodies or binding fragments or peptidomimetics thereof, RNA-based compounds, oligonucleotide-based compounds, and/or small molecule inhibitors of TLR7 activity or of a signaling protein's activity.

In the various methods according to the invention, a therapeutically or prophylactically effective amount of a synthetic oligonucleotide of the invention and effective in inhibiting the expression of TLR7 is administered to a cell. This cell may be part of a cell culture, a neovascularized tissue culture, or may be part or the whole body of a mammal such as a human or other mammal. Administration may be by any suitable route, including, without limitation, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form. Administration of the therapeutic compositions of TLR7 antisense oligonucleotide can be carried out using known procedures at dosages and for periods of time effective to reduce symptoms or surrogate markers of the disease, depending on the condition and response, as determined by those with skill in the art. It may be desirable to administer simultaneously, or sequentially a therapeutically effective amount of one or more of the therapeutic TLR7 antisense oligonucleotides of the invention to an individual as a single treatment episode. In some exemplar embodiments of the methods of the invention described above, the oligonucleotide is administered locally and/or systemically. The term “administered locally” refers to delivery to a defined area or region of the body, while the term “systemic administration” is meant to encompass delivery to the whole organism.

In any of the methods according to the invention, one or more of the TLR7 antisense oligonucleotide can be administered either alone or in combination with any other agent useful for treating the disease or condition that does not diminish the immune modulatory effect of the TLR7 antisense oligonucleotide. In any of the methods according to the invention, the agent useful for treating the disease or condition includes, but is not limited to, one or more vaccines, antigens, antibodies, cytotoxic agents, allergens, antibiotics, antisense oligonucleotides, TLR agonist, TLR antagonist, siRNA, miRNA, peptides, proteins, gene therapy vectors, DNA vaccines, adjuvants or kinase inhibitors to enhance the specificity or magnitude of the immune response, or co-stimulatory molecules such as cytokines, chemokines, protein ligands, trans-activating factors, peptides and peptides comprising modified amino acids. For example, in the treatment of autoimmune disease, it is contemplated that the TLR7 antisense oligonucleotide may be administered in combination with one or more targeted therapeutic agents and/or monoclonal antibodies. Alternatively, the agent can include DNA vectors encoding for antigen or allergen. In these embodiments, the TLR7 antisense oligonucleotide of the invention can produce direct immune modulatory or suppressive effects. When co-administered with one or more other therapies, the synthetic oligonucleotide of the invention may be administered either simultaneously with the other treatment(s), or sequentially.

In the various methods according to the invention the route of administration may be, without limitation, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% synthetic oligonucleotide and preferably from about 25 to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution or glycols such as ethylene glycol, propylene glycol or polyethylene glycol. When administered in liquid form, the pharmaceutical composition contains from about 0.5 to 90% by weight of the synthetic oligonucleotide or from about 1 to 50% synthetic oligonucleotide.

When a therapeutically effective amount of synthetic oligonucleotide of the invention is administered by parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form, the synthetic antisense oligonucleotide will be in the form of a pyrogen-free, parenterally acceptable aqueous solution. The preparation of such parenterally acceptable solutions, having due regard to pH, isotonicity, stability, and the like, is within the skill in the art. An pharmaceutical composition for parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection or other vehicle as known in the art. The pharmaceutical composition of the present invention may also contain stabilizers, preservatives, buffers, antioxidants or other additives known to those of skill in the art.

When administered parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form, doses ranging from 0.01% to 10% (weight/volume) may be used. When administered in liquid form, a liquid carrier such as water, petroleum, oils of animal or plant origin such as peanut oil, mineral oil, soybean oil, sesame oil or synthetic oils may be added. Topical administration may be by liposome or transdermal time-release patch.

The amount of synthetic oligonucleotide in the pharmaceutical composition of the present invention will depend upon the nature and severity of the condition being treated, and on the nature of prior treatments which the patent has undergone. It is contemplated that the various pharmaceutical compositions used to practice the method of the present invention should contain about 10 micrograms to about 20 mg of synthetic oligonucleotide per kg body or organ weight.

The duration of intravenous therapy using the pharmaceutical composition of the present invention will vary, depending on the severity of the disease being treated and the condition and potential idiosyncratic response of each individual patient.

Some diseases lend themselves to acute treatment while others require longer term therapy. Both acute and long term intervention in diseases are worthy goals. Injections of antisense oligonucleotides against TLR7 can be an effective means of inhibiting certain diseases in an acute situation. However for long term therapy over a period of weeks, months or years, systemic delivery (intraperitoneal, intramuscular, subcutaneous, intravenous) either with carriers such as saline, slow release polymers or liposomes are likely to be considered.

In some chronic diseases, systemic administration of oligonucleotides may be preferable. The frequency of injections is from continuous infusion to once a month, several times per month or less frequently will be determined based on the disease process and the biological half life of the oligonucleotides.

The oligonucleotides and methods of the invention are also useful for examining the function of the TLR7 gene in a cell or in a control mammal or in a mammal afflicted with a disease associated with TLR7 or immune stimulation through TLR7. In such use, the cell or mammal is administered the oligonucleotide, and the expression of TLR7 mRNA or protein is examined.

Without being limited to any theory or mechanism, it is generally believed that the activity of oligonucleotides according to the invention depends on the hybridization of the oligonucleotide to the target nucleic acid (e.g. to at least a portion of a genomic region, gene or mRNA transcript thereof), thus disrupting the function of the target. Such hybridization under physiological conditions is measured as a practical matter by observing interference with the function of the nucleic acid sequence. Thus, an exemplar oligonucleotide used in accordance with the invention is capable of forming a stable duplex (or triplex in the Hoogsteen or other hydrogen bond pairing mechanism) with the target nucleic acid; activating RNase H or other in vivo enzymes thereby causing effective destruction of the target RNA molecule; and is capable of resisting nucleolytic degradation (e.g. endonuclease and exonuclease activity) in vivo. A number of the modifications to oligonucleotides described above and others which are known in the art specifically and successfully address each of these exemplar characteristics.

In the various methods of treatment or use of the present invention, a therapeutically or prophylactically effective amount of one, two or more of the synthetic oligonucleotides of the invention is administered to a subject afflicted with or at risk of developing a disease or disorder. The antisense oligonucleotide(s) of the invention may be administered in accordance with the method of the invention either alone or in combination with other known therapies, including but not limited to, one or more vaccines, antigens, antibodies, cytotoxic agents, allergens, antibiotics, antisense oligonucleotides, TLR agonist, TLR antagonist, siRNA, miRNA, peptides, proteins, gene therapy vectors, DNA vaccines, adjuvants or kinase inhibitors to enhance the specificity or magnitude of the immune response, or co-stimulatory molecules such as cytokines, chemokines, protein ligands, trans-activating factors, peptides and peptides comprising modified amino acids. When co-administered with one or more other therapies, the synthetic oligonucleotide of the invention may be administered either simultaneously with the other treatment(s), or sequentially.

The following examples illustrate the exemplar modes of making and practicing the present invention, but are not meant to limit the scope of the invention since alternative methods may be utilized to obtain similar results.

### Example 1

### Preparation of TLR7-Specific Antisense Oligonucleotides

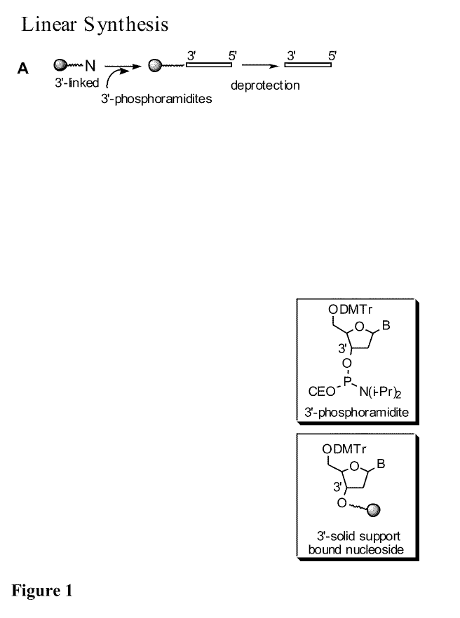

Chemical entities according to the invention were synthesized on a 1 μmol to 0.1 mM scale using an automated DNA synthesizer (OligoPilot II, AKTA, (Amersham) and/or Expedite 8909 (Applied Biosystem), following the linear synthesis procedure outlined in FIG. 1.

5′-DMT dA, dG, dC and T phosphoramidites were purchased from Proligo (Boulder, Colo.). 5′-DMT 7-deaza-dG and araG phosphoramidites were obtained from Chemgenes (Wilmington, Mass.). DiDMT-glycerol linker solid support was obtained from Chemgenes. 1-(2′-deoxy-β-D-ribofuranosyl)-2-oxo-7-deaza-8-methyl-purine amidite was obtained from Glen Research (Sterling, Va.), 2′-O-methylribonuncleoside amidites were obtained from Promega (Obispo, Calif.). All compounds according to the invention were phosphorothioate backbone modified.

All nucleoside phosphoramidites were characterized by ³¹P and ¹H NMR spectra. Modified nucleosides were incorporated at specific sites using normal coupling cycles recommended by the supplier. After synthesis, compounds were deprotected using concentrated ammonium hydroxide and purified by reverse phase HPLC, detritylation, followed by dialysis. Purified compounds as sodium salt form were lyophilized prior to use. Purity was tested by CGE and MALDI-TOF MS. Endotoxin levels were determined by LAL test and were below 1.0 EU/mg.

### Example 2

### Cell Culture Conditions and Reagents

### HEK293 Cell Culture Assays for TLR7 Antisense Activity

HEK293 XL cells stably expressing human TLR7 (Invivogen, San Diego, Calif.) were plated in 48-well plates in 250 μL/well DMEM supplemented with 10% heat-inactivated FBS in a 5% CO2 incubator. At 80% confluence, cultures were transiently transfected with 400 ng/mL of the secreted form of human embryonic alkaline phosphatase (SEAP) reporter plasmid (pNifty2-Seap) (Invivogen) in the presence of 4 μL/mL of lipofectamine (Invitrogen, Carlsbad, Calif.) in culture medium. Plasmid DNA and lipofectamine were diluted separately in serum-free medium and incubated at room temperature for 5 min. After incubation, the diluted DNA and lipofectamine were mixed and the mixtures were incubated further at room temperature for 20 min. Aliquots of 25 μL of the DNA/lipofectamine mixture containing 100 ng of plasmid DNA and 1 μL of lipofectamine were added to each well of the cell culture plate, and the cells were transfected for 6 h. After transfection, medium was replaced with fresh culture medium (no antibiotics), antisense compounds were added to the wells, and incubation continued for 18-20 h. Cells were then stimulated with the TLR7 agonist for 24 h.

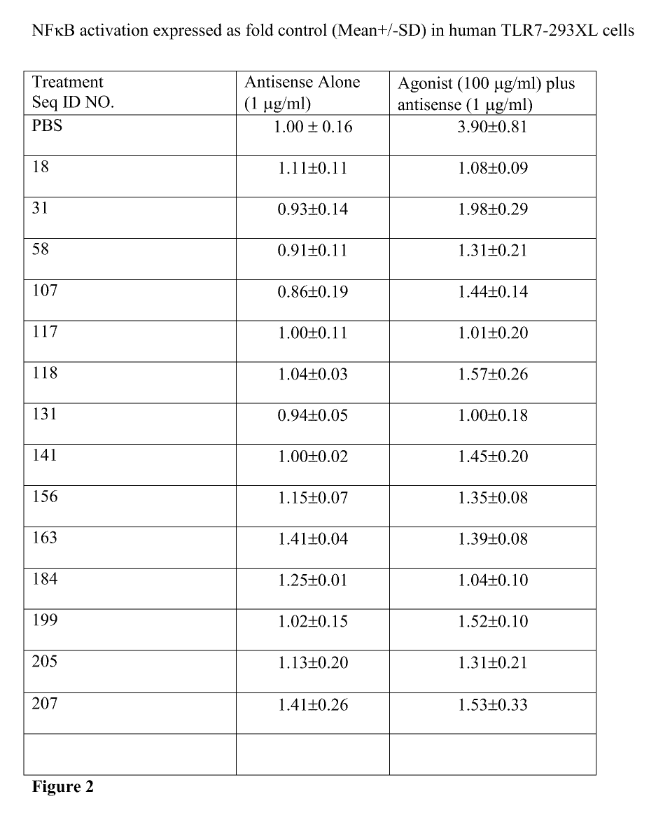

At the end of the treatment, 20 μL of culture supernatant was taken from each well and assayed for SEAP assay by the Quanti Blue method according to the manufacturer's protocol (Invivogen). The data are shown in FIG. 2 as fold increase in NF-κB activity over PBS control.

### Example 3

### In Vivo Activity of TLR7 Antisense Oligonucleotide

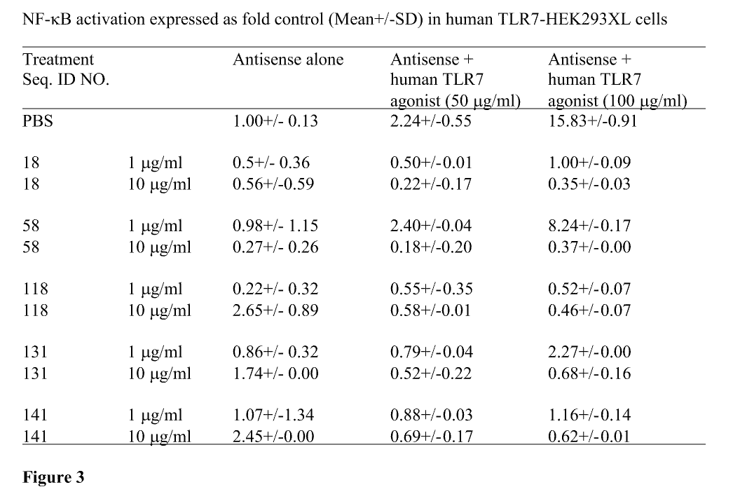

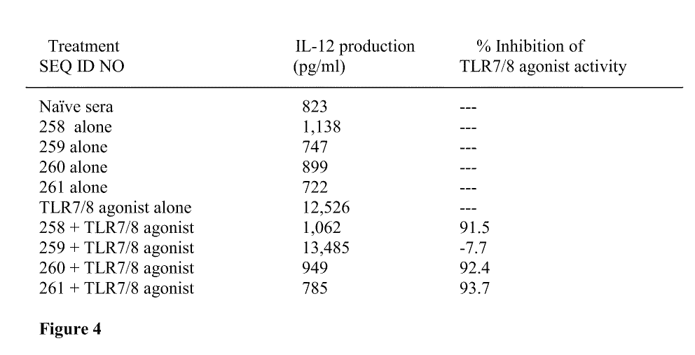

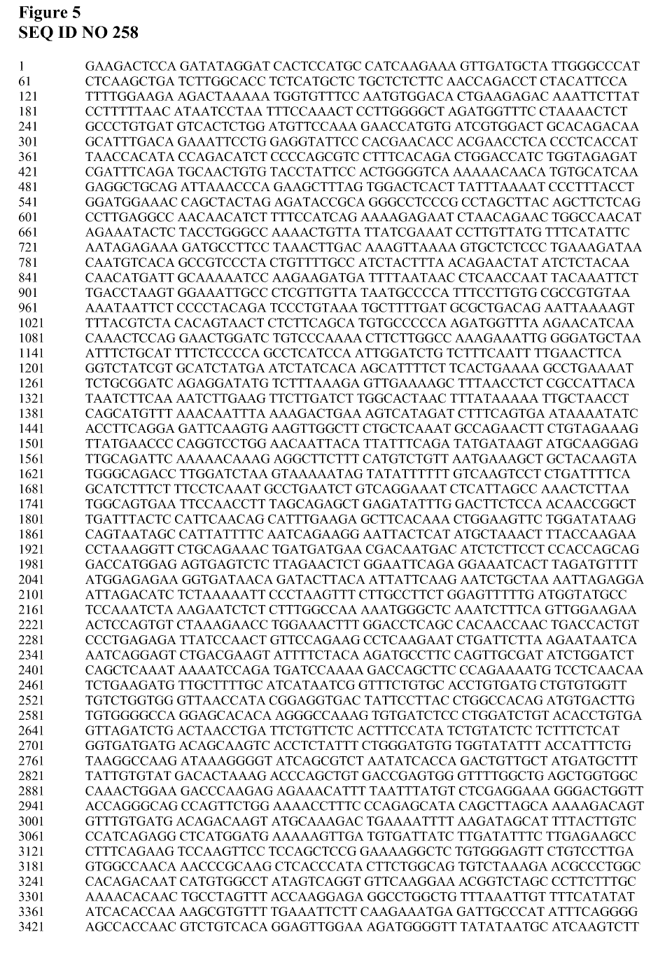

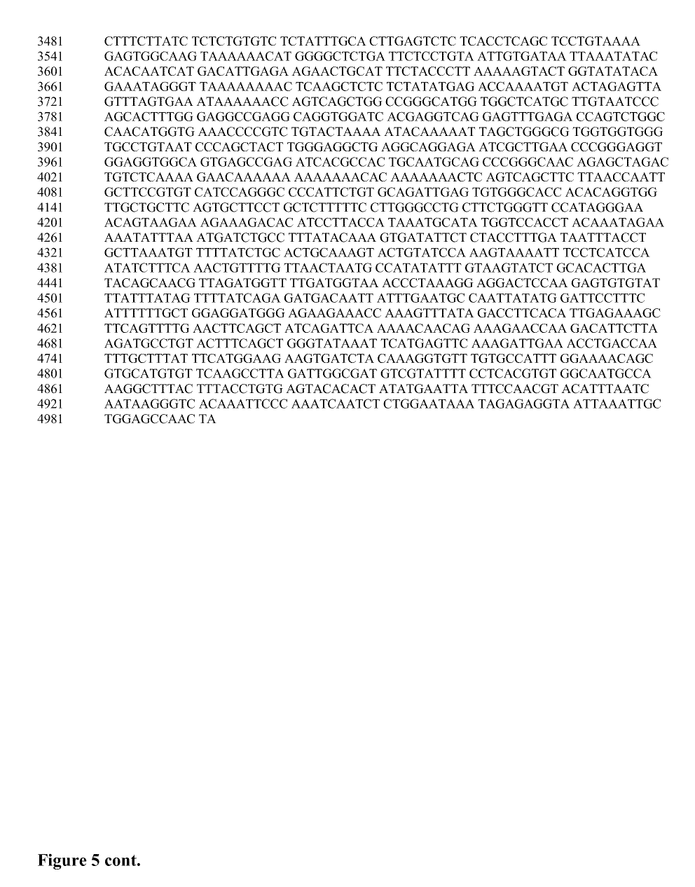

Female C57BL/6 mice of 5-6 weeks age (N=3/group) were injected with exemplar TLR7 antisense oligonucleotides according to the invention at 5 mg/kg, or PBS, subcutaneously once a day for three days. Subsequent to administration of the TLR7 antisense oligonucleotide, mice were injected with 0.25 mg/kg of a TLR7 agonist subcutaneously. Two hours after administration of the TLR7 agonist, blood was collected and IL-12 concentration was determined by ELISA, as shown in FIG. 3.

### EQUIVALENTS

Those skilled in the art will recognize, or be able to ascertain, using no more than routine experimentation, numerous equivalents to the specific substances and procedures described herein. For example, antisense oligonucleotides that overlap with the oligonucleotides may be used. Such equivalents are considered to be within the scope of this invention, and are covered by the following claims.

1. A synthetic antisense oligonucleotide 20 to 50 nucleotides in length targeted to TLR7 mRNA (SEQ ID NO: 258), wherein the antisense oligonucleotide has a sequence comprising SEQ ID NOs: 18, 31, 58, 107, 117, 118, 131, 141, 156, 163, 184, 199, 205 or 207, and wherein the oligonucleotide specifically hybridizes to and inhibits the expression of human TLR7.

2-5. (canceled)

6. A composition comprising a synthetic antisense oligonucleotide according to claim 1 and a physiologically acceptable carrier.

7. A method for inhibiting the expression of TLR7, the method comprising administering a synthetic antisense oligonucleotide according to claim 1.

8. A method for inhibiting the expression of TLR7, the method comprising administering a composition according to claim 6.

9. A method for inhibiting the expression of TLR7 in a mammal, the method comprising administering to the mammal a synthetic antisense oligonucleotide according to claim 1.

10. A method for inhibiting the expression of TLR7 in a mammal, the method comprising administering to the mammal a composition according to claim 6.

11. A method for inhibiting a TLR7-mediated immune response in a mammal, the method comprising administering to the mammal a synthetic antisense oligonucleotide according to claim 1 in a pharmaceutically effective amount.

12. A method for inhibiting a TLR7-mediated immune response in a mammal, the method comprising administering to the mammal a composition according to claim 6 in a pharmaceutically effective amount.

13. A method for therapeutically treating a mammal having one or more diseases mediated by TLR7, the method comprising administering to the mammal a synthetic antisense oligonucleotide according to claim 1 in a pharmaceutically effective amount.

14. A method for therapeutically treating a mammal having one or more diseases mediated by TLR7, the method comprising administering to the mammal a composition according to claim 6 in a pharmaceutically effective amount.

15. A method for preventing in a mammal one or more diseases or disorders mediated by TLR7, the method comprising administering to the mammal a synthetic antisense oligonucleotide according to claim 1 in a prophylactically effective amount.

16. A method for preventing in a mammal one or more diseases or disorders mediated by TLR7, the method comprising administering to the mammal a composition according to claim 6 in a prophylactically effective amount.

17. A method for down-regulating TLR7 expression and thus preventing undesired TLR7-mediated immune stimulation by a compound that activates TLR7, the method comprising administering a synthetic antisense oligonucleotide according to claim 1 in combination with one or more compounds which comprise an immunostimulatory motif that would activate a TLR7-mediated immune response but for the presence the antisense oligonucleotide.

18. A method for down-regulating TLR7 expression and thus preventing undesired TLR7-mediated immune stimulation by a compound that activates TLR7, the method comprising administering a composition according to claim 6 in combination with one or more compounds which comprise an immunostimulatory motif that would activate a TLR7-mediated immune response but for the presence of the composition.

19. The method according to claim 9, wherein the mammal is a human.

20. The method according to claim 13, wherein the one or more diseases are selected from the group consisting of cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma and a disease caused by a pathogen.

21. The method according to claim 20, wherein the autoimmune disorder is selected from the group consisting of lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis.

22. The method according to claim 20, wherein the inflammatory disorder is selected from the group consisting of airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

23. The method according to claim 17, wherein the compound is one or more non-TLR7 antisense oligonucleotides comprising an immunostimulatory motif that would otherwise activate a TLR7-mediated immune response.

24. The method according to claim 7, wherein the route of administration is selected from the group consisting of parenteral, intramuscular, subcutaneous, intraperitoneal, intraveneous, mucosal delivery, oral, sublingual, transdermal, topical, inhalation, intranasal, aerosol, intraocular, intratracheal, intrarectal, vaginal, gene gun, dermal patch, eye drop and mouthwash.

25. The method according to of claim 7, comprising further administering one or more vaccines, antigens, antibodies, cytotoxic agents, allergens, antibiotics, antisense oligonucleotides, TLR agonist, TLR antagonist, siRNA, miRNA, antisense oligonucleotides, aptamers, proteins, gene therapy vectors, DNA vaccines, adjuvants, co-stimulatory molecules or combinations thereof.

26. A method for inhibiting TLR7 expression and activity in a mammal, comprising administering to the mammal an antisense oligonucleotide complementary to TLR7 mRNA and an antagonist of TLR7 protein.

27. The method according to claim 26, wherein the TLR 7 protein antagonist is selected from the group consisting of anti-TLR7 antibodies or binding fragments or peptidomimetics thereof, RNA-based compounds, oligonucleotide-based compounds, and small molecule inhibitors of TLR7 activity.

28. The method according to claim 15, wherein the one or more diseases are selected from the group consisting of cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma and a disease caused by a pathogen.

29. The method according to claim 28, wherein the autoimmune disorder is selected from the group consisting of lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis.

30. The method according to claim 28, wherein the inflammatory disorder is selected from the group consisting of airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.


SEQUENCE LISTING (262 sequences)

SEQ ID NO: 1 - DNA, 20 bp, Artificial Sequence
  Antisense Oligonucleotide
atcctatatctggagtcttc

SEQ ID NO: 2 - DNA, 20 bp, Artificial Sequence
  Antisense Oligonucleotide
ttgatggcatggagtgatcc

SEQ ID NO: 3 - DNA, 20 bp, Artificial Sequence
  Antisense Oligonucleotide
tttcttgatggcatggagtg

SEQ ID NO: 4 - DNA, 20 bp, Artificial Sequence
  Antisense Oligonucleotide
atgggcccaatagcatcaac

SEQ ID NO: 5 - DNA, 20 bp, Artificial Sequence
  Antisense Oligonucleotide
ggtgccaagatcagcttgag

SEQ ID NO: 6 - DNA, 20 bp, Artificial Sequence
  Antisense Oligonucleotide
agcagagcatgagaggtgcc

SEQ ID NO: 7 - DNA, 20 bp, Artificial Sequence
  Antisense Oligonucleotide
gaagagagcagagcatgaga

SEQ ID NO: 8 - DNA, 20 bp, Artificial Sequence
  Antisense Oligonucleotide
tggaatgtagaggtctggtt

SEQ ID NO: 9 - DNA, 20 bp, Artificial Sequence
  Antisense Oligonucleotide
tttttagtcttcttccaaaa

SEQ ID NO: 10 - DNA, 20 bp, Artificial Sequence
  Antisense Oligonucleotide
tgtccacattggaaacacca

SEQ ID NO: 11 - DNA, 20 bp, Artificial Sequence
  Antisense Oligonucleotide
tttgtctcttcagtgtccac

SEQ ID NO: 12 - DNA, 20 bp, Artificial Sequence
  Antisense Oligonucleotide
ataagaatttgtctcttcag

SEQ ID NO: 13 - DNA, 20 bp, Artificial Sequence
  Antisense Oligonucleotide
ttaggattatgttaaaaagg

SEQ ID NO: 14 - DNA, 20 bp, Artificial Sequence
  Antisense Oligonucleotide
agccccaaggagtttggaaa

SEQ ID NO: 15 - DNA, 20 bp, Artificial Sequence
  Antisense Oligonucleotide
agagttttaggaaaccatct

SEQ ID NO: 16 - DNA, 20 bp, Artificial Sequence
  Antisense Oligonucleotide
ccagagtgacatcacagggc

SEQ ID NO: 17 - DNA, 20 bp, Artificial Sequence
  Antisense Oligonucleotide
cacatggttctttggaacat

SEQ ID NO: 18 - DNA, 20 bp, Artificial Sequence
  Antisense Oligonucleotide
tgcttgtctgtgcagtccac

SEQ ID NO: 19 - DNA, 20 bp, Artificial Sequence
  Antisense Oligonucleotide
caggaatttctgtcaaatgc

SEQ ID NO: 20 - DNA, 20 bp, Artificial Sequence
  Antisense Oligonucleotide
ggtgttcgtgggaatacctc

SEQ ID NO: 21 - DNA, 20 bp, Artificial Sequence
  Antisense Oligonucleotide
atggtgagggtgaggttcgt

SEQ ID NO: 22 - DNA, 20 bp, Artificial Sequence
  Antisense Oligonucleotide
agatgtctggtatgtggtta

SEQ ID NO: 23 - DNA, 20 bp, Artificial Sequence
  Antisense Oligonucleotide
tctgtgaaaggacgctgggg

SEQ ID NO: 24 - DNA, 20 bp, Artificial Sequence
  Antisense Oligonucleotide
atctctaccagatggtccag

SEQ ID NO: 25 - DNA, 20 bp, Artificial Sequence
  Antisense Oligonucleotide
cacagttgcatctgaaatcg

SEQ ID NO: 26 - DNA, 20 bp, Artificial Sequence
  Antisense Oligonucleotide
tgaccccagtggaataggta

SEQ ID NO: 27 - DNA, 20 bp, Artificial Sequence
  Antisense Oligonucleotide
ttgatgcacatgttgttttt

SEQ ID NO: 28 - DNA, 20 bp, Artificial Sequence
  Antisense Oligonucleotide
tgggtttaatctgcagcctc

SEQ ID NO: 29 - DNA, 20 bp, Artificial Sequence
  Antisense Oligonucleotide
agtgagtccactaaagcttc

SEQ ID NO: 30 - DNA, 20 bp, Artificial Sequence
  Antisense Oligonucleotide
aggtaaagggattttaaata

SEQ ID NO: 31 - DNA, 20 bp, Artificial Sequence
  Antisense Oligonucleotide
ctagtagctggtttccatcc

SEQ ID NO: 32 - DNA, 20 bp, Artificial Sequence
  Antisense Oligonucleotide
cgggaggccctgcggtatct

SEQ ID NO: 33 - DNA, 20 bp, Artificial Sequence
  Antisense Oligonucleotide
ctgagaagctgtaagctagg

SEQ ID NO: 34 - DNA, 20 bp, Artificial Sequence
  Antisense Oligonucleotide
agatgttgttggcctcaagg

SEQ ID NO: 35 - DNA, 20 bp, Artificial Sequence
  Antisense Oligonucleotide
attctcttttctgatggaaa

SEQ ID NO: 36 - DNA, 20 bp, Artificial Sequence
  Antisense Oligonucleotide
atgttggccagttctgttag

SEQ ID NO: 37 - DNA, 20 bp, Artificial Sequence
  Antisense Oligonucleotide
ggcccaggtagagtatttct

SEQ ID NO: 38 - DNA, 20 bp, Artificial Sequence
  Antisense Oligonucleotide
atttcgataataacagtttt

SEQ ID NO: 39 - DNA, 20 bp, Artificial Sequence
  Antisense Oligonucleotide
gaatatgaaacataacaagg

SEQ ID NO: 40 - DNA, 20 bp, Artificial Sequence
  Antisense Oligonucleotide
ggaaggcatctttctctatt

SEQ ID NO: 41 - DNA, 20 bp, Artificial Sequence
  Antisense Oligonucleotide
ttttaactttgtcaagttta

SEQ ID NO: 42 - DNA, 20 bp, Artificial Sequence
  Antisense Oligonucleotide
ttatctttcagggagagcac

SEQ ID NO: 43 - DNA, 20 bp, Artificial Sequence
  Antisense Oligonucleotide
tagggacggctgtgacattg

SEQ ID NO: 44 - DNA, 20 bp, Artificial Sequence
  Antisense Oligonucleotide
taaagtagatggcaaaacag

SEQ ID NO: 45 - DNA, 20 bp, Artificial Sequence
  Antisense Oligonucleotide
ttgtagagatatagttctgt

SEQ ID NO: 46 - DNA, 20 bp, Artificial Sequence
  Antisense Oligonucleotide
ggatttttgcaatcatgttg

SEQ ID NO: 47 - DNA, 20 bp, Artificial Sequence
  Antisense Oligonucleotide
gttattaaaatcatcttctt

SEQ ID NO: 48 - DNA, 20 bp, Artificial Sequence
  Antisense Oligonucleotide
agaatttgtaattggttgag

SEQ ID NO: 49 - DNA, 20 bp, Artificial Sequence
  Antisense Oligonucleotide
ggcaatttccacttaggtca

SEQ ID NO: 50 - DNA, 20 bp, Artificial Sequence
  Antisense Oligonucleotide
tggggcattataacaacgag

SEQ ID NO: 51 - DNA, 20 bp, Artificial Sequence
  Antisense Oligonucleotide
ttacacggcgcacaaggaaa

SEQ ID NO: 52 - DNA, 20 bp, Artificial Sequence
  Antisense Oligonucleotide
tctgtaggggagaattattt

SEQ ID NO: 53 - DNA, 20 bp, Artificial Sequence
  Antisense Oligonucleotide
atcaaaagcatttacaggga

SEQ ID NO: 54 - DNA, 20 bp, Artificial Sequence
  Antisense Oligonucleotide
acttttaattctgtcagcgc

SEQ ID NO: 55 - DNA, 20 bp, Artificial Sequence
  Antisense Oligonucleotide
agttactgtgtagacgtaaa

SEQ ID NO: 56 - DNA, 20 bp, Artificial Sequence
  Antisense Oligonucleotide
tgggggcacatgctgaagag

SEQ ID NO: 57 - DNA, 20 bp, Artificial Sequence
  Antisense Oligonucleotide
ttgatgttcttaaaccatct

SEQ ID NO: 58 - DNA, 20 bp, Artificial Sequence
  Antisense Oligonucleotide
cctggagtttgttgatgttc

SEQ ID NO: 59 - DNA, 20 bp, Artificial Sequence
  Antisense Oligonucleotide
gatccagttcctggagtttg

SEQ ID NO: 60 - DNA, 20 bp, Artificial Sequence
  Antisense Oligonucleotide
ggccaagaagttttgggaca

SEQ ID NO: 61 - DNA, 20 bp, Artificial Sequence
  Antisense Oligonucleotide
ttagcatccccaatttcttt

SEQ ID NO: 62 - DNA, 20 bp, Artificial Sequence
  Antisense Oligonucleotide
tggggagaaaatgcagaaat

SEQ ID NO: 63 - DNA, 20 bp, Artificial Sequence
  Antisense Oligonucleotide
cagatccaattggatgaggc

SEQ ID NO: 64 - DNA, 20 bp, Artificial Sequence
  Antisense Oligonucleotide
tgaagttcaaaattgaaaga

SEQ ID NO: 65 - DNA, 20 bp, Artificial Sequence
  Antisense Oligonucleotide
tcatagatgcacgatagacc

SEQ ID NO: 66 - DNA, 20 bp, Artificial Sequence
  Antisense Oligonucleotide
agaaaatgcttgtgatagat

SEQ ID NO: 67 - DNA, 20 bp, Artificial Sequence
  Antisense Oligonucleotide
attttcaggcttttcagtga

SEQ ID NO: 68 - DNA, 20 bp, Artificial Sequence
  Antisense Oligonucleotide
catatcctctgatccgcaga

SEQ ID NO: 69 - DNA, 20 bp, Artificial Sequence
  Antisense Oligonucleotide
gcttttcaactctttaaaga

SEQ ID NO: 70 - DNA, 20 bp, Artificial Sequence
  Antisense Oligonucleotide
tgtaatggcgagaggttaaa

SEQ ID NO: 71 - DNA, 20 bp, Artificial Sequence
  Antisense Oligonucleotide
cttcaagattttgaagatta

SEQ ID NO: 72 - DNA, 20 bp, Artificial Sequence
  Antisense Oligonucleotide
gttagtgccaagatcaagaa

SEQ ID NO: 73 - DNA, 20 bp, Artificial Sequence
  Antisense Oligonucleotide
aggttagcaatttttataaa

SEQ ID NO: 74 - DNA, 20 bp, Artificial Sequence
  Antisense Oligonucleotide
taaattgtttaaacatgctg

SEQ ID NO: 75 - DNA, 20 bp, Artificial Sequence
  Antisense Oligonucleotide
atctatgactttcagtcttt

SEQ ID NO: 76 - DNA, 20 bp, Artificial Sequence
  Antisense Oligonucleotide
gatattttattcactgaaag

SEQ ID NO: 77 - DNA, 20 bp, Artificial Sequence
  Antisense Oligonucleotide
cacttgaatctcctgaaggt

SEQ ID NO: 78 - DNA, 20 bp, Artificial Sequence
  Antisense Oligonucleotide
tctggcatttgagcagaagc

SEQ ID NO: 79 - DNA, 20 bp, Artificial Sequence
  Antisense Oligonucleotide
ctttctacagaagttctggc

SEQ ID NO: 80 - DNA, 20 bp, Artificial Sequence
  Antisense Oligonucleotide
ccaggacctggggttcataa

SEQ ID NO: 81 - DNA, 20 bp, Artificial Sequence
  Antisense Oligonucleotide
tctgaaataatgtaattgtt

SEQ ID NO: 82 - DNA, 20 bp, Artificial Sequence
  Antisense Oligonucleotide
ctccttgcatacttatcata

SEQ ID NO: 83 - DNA, 20 bp, Artificial Sequence
  Antisense Oligonucleotide
ctttgtttttgaatctgcaa

SEQ ID NO: 84 - DNA, 20 bp, Artificial Sequence
  Antisense Oligonucleotide
aacagacatgaaagaagcct

SEQ ID NO: 85 - DNA, 20 bp, Artificial Sequence
  Antisense Oligonucleotide
tacttgtagcagctttcatt

SEQ ID NO: 86 - DNA, 20 bp, Artificial Sequence
  Antisense Oligonucleotide
ttagatccaaggtctgccca

SEQ ID NO: 87 - DNA, 20 bp, Artificial Sequence
  Antisense Oligonucleotide
aaaaaatatactatttttac

SEQ ID NO: 88 - DNA, 20 bp, Artificial Sequence
  Antisense Oligonucleotide
tgaaaatcagaggacttgac

SEQ ID NO: 89 - DNA, 20 bp, Artificial Sequence
  Antisense Oligonucleotide
atttgaggaaagaaagatgc

SEQ ID NO: 90 - DNA, 20 bp, Artificial Sequence
  Antisense Oligonucleotide
atttcctgacagattcaggc

SEQ ID NO: 91 - DNA, 20 bp, Artificial Sequence
  Antisense Oligonucleotide
ttaagagtttggctaatgag

SEQ ID NO: 92 - DNA, 20 bp, Artificial Sequence
  Antisense Oligonucleotide
aaggttggaattcactgcca

SEQ ID NO: 93 - DNA, 20 bp, Artificial Sequence
  Antisense Oligonucleotide
caaatatctcagctctgcta

SEQ ID NO: 94 - DNA, 20 bp, Artificial Sequence
  Antisense Oligonucleotide
agccggttgttggagaagtc

SEQ ID NO: 95 - DNA, 20 bp, Artificial Sequence
  Antisense Oligonucleotide
ctgttgaatggagtaaatca

SEQ ID NO: 96 - DNA, 20 bp, Artificial Sequence
  Antisense Oligonucleotide
tttgtgaagctcttcaaatg

SEQ ID NO: 97 - DNA, 20 bp, Artificial Sequence
  Antisense Oligonucleotide
cttatatccagaacttccag

SEQ ID NO: 98 - DNA, 20 bp, Artificial Sequence
  Antisense Oligonucleotide
gaaaataatggctattactg

SEQ ID NO: 99 - DNA, 20 bp, Artificial Sequence
  Antisense Oligonucleotide
atgagtaattccttctgatt

SEQ ID NO: 100 - DNA, 20 bp, Artificial Sequence
  Antisense Oligonucleotide
ttcttggtaaagtttagcat

SEQ ID NO: 101 - DNA, 20 bp, Artificial Sequence
  Antisense Oligonucleotide
gtttctgcagaacctttagg

SEQ ID NO: 102 - DNA, 20 bp, Artificial Sequence
  Antisense Oligonucleotide
gtcattgtcgttcatcatca

SEQ ID NO: 103 - DNA, 20 bp, Artificial Sequence
  Antisense Oligonucleotide
ctgctggtggaggaagagat

SEQ ID NO: 104 - DNA, 20 bp, Artificial Sequence
  Antisense Oligonucleotide
gagactcactctccatggtc

SEQ ID NO: 105 - DNA, 20 bp, Artificial Sequence
  Antisense Oligonucleotide
tctgaattccagagttctaa

SEQ ID NO: 106 - DNA, 20 bp, Artificial Sequence
  Antisense Oligonucleotide
aaaacatctaagtgatttcc

SEQ ID NO: 107 - DNA, 20 bp, Artificial Sequence
  Antisense Oligonucleotide
tctgttatcaccttctctcc

SEQ ID NO: 108 - DNA, 20 bp, Artificial Sequence
  Antisense Oligonucleotide
cttgaataattgtaagtatc

SEQ ID NO: 109 - DNA, 20 bp, Artificial Sequence
  Antisense Oligonucleotide
tcctctaattttagcagatt

SEQ ID NO: 110 - DNA, 20 bp, Artificial Sequence
  Antisense Oligonucleotide
aatttttagagatgtctaat

SEQ ID NO: 111 - DNA, 20 bp, Artificial Sequence
  Antisense Oligonucleotide
agaaggcaagaaacttaggg

SEQ ID NO: 112 - DNA, 20 bp, Artificial Sequence
  Antisense Oligonucleotide
ggcataccatcaaaaactcc

SEQ ID NO: 113 - DNA, 20 bp, Artificial Sequence
  Antisense Oligonucleotide
agagattctttagatttgga

SEQ ID NO: 114 - DNA, 20 bp, Artificial Sequence
  Antisense Oligonucleotide
gagcccatttttggccaaag

SEQ ID NO: 115 - DNA, 20 bp, Artificial Sequence
  Antisense Oligonucleotide
ttcttccaactgaaagattt

SEQ ID NO: 116 - DNA, 20 bp, Artificial Sequence
  Antisense Oligonucleotide
ggttctttagacactggagt

SEQ ID NO: 117 - DNA, 20 bp, Artificial Sequence
  Antisense Oligonucleotide
ggtccaaagtttccaggttc

SEQ ID NO: 118 - DNA, 20 bp, Artificial Sequence
  Antisense Oligonucleotide
gttggttgtggctgaggtcc

SEQ ID NO: 119 - DNA, 20 bp, Artificial Sequence
  Antisense Oligonucleotide
acagtggtcagttggttgtg

SEQ ID NO: 120 - DNA, 20 bp, Artificial Sequence
  Antisense Oligonucleotide
agttggataatctctcaggg

SEQ ID NO: 121 - DNA, 20 bp, Artificial Sequence
  Antisense Oligonucleotide
attcttgaggcttctggaac

SEQ ID NO: 122 - DNA, 20 bp, Artificial Sequence
  Antisense Oligonucleotide
tgattattcttaagaatcag

SEQ ID NO: 123 - DNA, 20 bp, Artificial Sequence
  Antisense Oligonucleotide
acttcgtcagactcctgatt

SEQ ID NO: 124 - DNA, 20 bp, Artificial Sequence
  Antisense Oligonucleotide
gaaggcatcttgtagaaaat

SEQ ID NO: 125 - DNA, 20 bp, Artificial Sequence
  Antisense Oligonucleotide
agatccagatatcgcaactg

SEQ ID NO: 126 - DNA, 20 bp, Artificial Sequence
  Antisense Oligonucleotide
tctggattttatttgagctg

SEQ ID NO: 127 - DNA, 20 bp, Artificial Sequence
  Antisense Oligonucleotide
gaagctggtcttttggatca

SEQ ID NO: 128 - DNA, 20 bp, Artificial Sequence
  Antisense Oligonucleotide
ttgttgaggacattttctgg

SEQ ID NO: 129 - DNA, 20 bp, Artificial Sequence
  Antisense Oligonucleotide
gcaaaagcaacatcttcaga

SEQ ID NO: 130 - DNA, 20 bp, Artificial Sequence
  Antisense Oligonucleotide
gcacagaaaccgattatgat

SEQ ID NO: 131 - DNA, 20 bp, Artificial Sequence
  Antisense Oligonucleotide
ccacacagcatcacaggtgc

SEQ ID NO: 132 - DNA, 20 bp, Artificial Sequence
  Antisense Oligonucleotide
tatggttaacccaccagaca

SEQ ID NO: 133 - DNA, 20 bp, Artificial Sequence
  Antisense Oligonucleotide
gtaaggaatagtcacctccg

SEQ ID NO: 134 - DNA, 20 bp, Artificial Sequence
  Antisense Oligonucleotide
caagtcacatctgtggccag

SEQ ID NO: 135 - DNA, 20 bp, Artificial Sequence
  Antisense Oligonucleotide
tgtgtgctcctggccccaca

SEQ ID NO: 136 - DNA, 20 bp, Artificial Sequence
  Antisense Oligonucleotide
ggagatcacactttggccct

SEQ ID NO: 137 - DNA, 20 bp, Artificial Sequence
  Antisense Oligonucleotide
tcacaggtgtacagatccag

SEQ ID NO: 138 - DNA, 20 bp, Artificial Sequence
  Antisense Oligonucleotide
tcaggttagtcagatctaac

SEQ ID NO: 139 - DNA, 20 bp, Artificial Sequence
  Antisense Oligonucleotide
tatggaaagtgagaacagaa

SEQ ID NO: 140 - DNA, 20 bp, Artificial Sequence
  Antisense Oligonucleotide
atgagaaagagagatacaga

SEQ ID NO: 141 - DNA, 20 bp, Artificial Sequence
  Antisense Oligonucleotide
gacttgctgtcatcatcacc

SEQ ID NO: 142 - DNA, 20 bp, Artificial Sequence
  Antisense Oligonucleotide
cacatcccagaaatagaggt

SEQ ID NO: 143 - DNA, 20 bp, Artificial Sequence
  Antisense Oligonucleotide
cagaaatggtaaatatacca

SEQ ID NO: 144 - DNA, 19 bp, Artificial Sequence
  Antisense Oligonucleotide
acccctttacttggcctta

SEQ ID NO: 145 - DNA, 20 bp, Artificial Sequence
  Antisense Oligonucleotide
tggtgatattagacgctgat

SEQ ID NO: 146 - DNA, 20 bp, Artificial Sequence
  Antisense Oligonucleotide
aaagcatcatagcaacagtc

SEQ ID NO: 147 - DNA, 20 bp, Artificial Sequence
  Antisense Oligonucleotide
ctttagtgtcatacacaata

SEQ ID NO: 148 - DNA, 20 bp, Artificial Sequence
  Antisense Oligonucleotide
ccactcggtcacagctgggt

SEQ ID NO: 149 - DNA, 20 bp, Artificial Sequence
  Antisense Oligonucleotide
gccaccagctcagccaaaac

SEQ ID NO: 150 - DNA, 20 bp, Artificial Sequence
  Antisense Oligonucleotide
ctcttgggtcttccagtttg

SEQ ID NO: 151 - DNA, 20 bp, Artificial Sequence
  Antisense Oligonucleotide
acataaattaaaatgtttct

SEQ ID NO: 152 - DNA, 20 bp, Artificial Sequence
  Antisense Oligonucleotide
aaccagtccctttcctcgag

SEQ ID NO: 153 - DNA, 20 bp, Artificial Sequence
  Antisense Oligonucleotide
ccagaactggctgccctggt

SEQ ID NO: 154 - DNA, 20 bp, Artificial Sequence
  Antisense Oligonucleotide
tatgctctgggaaaggtttt

SEQ ID NO: 155 - DNA, 20 bp, Artificial Sequence
  Antisense Oligonucleotide
actgtctttttgctaagctg

SEQ ID NO: 156 - DNA, 20 bp, Artificial Sequence
  Antisense Oligonucleotide
gcatacttgtctgtcatcac

SEQ ID NO: 157 - DNA, 20 bp, Artificial Sequence
  Antisense Oligonucleotide
aaaattttcagtctttgcat

SEQ ID NO: 158 - DNA, 20 bp, Artificial Sequence
  Antisense Oligonucleotide
gacaagtaaaatgctatctt

SEQ ID NO: 159 - DNA, 20 bp, Artificial Sequence
  Antisense Oligonucleotide
catccatgagcctctgatgg

SEQ ID NO: 160 - DNA, 20 bp, Artificial Sequence
  Antisense Oligonucleotide
gataatcacatcaacttttt

SEQ ID NO: 161 - DNA, 20 bp, Artificial Sequence
  Antisense Oligonucleotide
ggcttctcaagaaatatcaa

SEQ ID NO: 162 - DNA, 20 bp, Artificial Sequence
  Antisense Oligonucleotide
ggaacttggacttctgaaag

SEQ ID NO: 163 - DNA, 20 bp, Artificial Sequence
  Antisense Oligonucleotide
gctggaggaacttggacttc

SEQ ID NO: 164 - DNA, 20 bp, Artificial Sequence
  Antisense Oligonucleotide
gagccttttccggagctgga

SEQ ID NO: 165 - DNA, 20 bp, Artificial Sequence
  Antisense Oligonucleotide
tcaaggacagaactcccaca

SEQ ID NO: 166 - DNA, 20 bp, Artificial Sequence
  Antisense Oligonucleotide
cttgcgggtttgttggccac

SEQ ID NO: 167 - DNA, 20 bp, Artificial Sequence
  Antisense Oligonucleotide
ctgccagaagtatgggtgag

SEQ ID NO: 168 - DNA, 20 bp, Artificial Sequence
  Antisense Oligonucleotide
gccagggcgttctttagaca

SEQ ID NO: 169 - DNA, 20 bp, Artificial Sequence
  Antisense Oligonucleotide
aggccacatgattgtctgtg

SEQ ID NO: 170 - DNA, 20 bp, Artificial Sequence
  Antisense Oligonucleotide
ttccttgaacacctgactat

SEQ ID NO: 171 - DNA, 20 bp, Artificial Sequence
  Antisense Oligonucleotide
gcaaagaagggctagaccgt

SEQ ID NO: 172 - DNA, 20 bp, Artificial Sequence
  Antisense Oligonucleotide
aaactaggcagttgtgtttt

SEQ ID NO: 173 - DNA, 20 bp, Artificial Sequence
  Antisense Oligonucleotide
cagccaggcctctccttggt

SEQ ID NO: 174 - DNA, 20 bp, Artificial Sequence
  Antisense Oligonucleotide
atatatgaaaacaatttaaa

SEQ ID NO: 175 - DNA, 20 bp, Artificial Sequence
  Antisense Oligonucleotide
aaacacgcttttggtgtgat

SEQ ID NO: 176 - DNA, 20 bp, Artificial Sequence
  Antisense Oligonucleotide
tcatttcttgaagaatttca

SEQ ID NO: 177 - DNA, 20 bp, Artificial Sequence
  Antisense Oligonucleotide
cccctgaaatatgggcaatc

SEQ ID NO: 178 - DNA, 20 bp, Artificial Sequence
  Antisense Oligonucleotide
tgtgacagacgttggtggct

SEQ ID NO: 179 - DNA, 20 bp, Artificial Sequence
  Antisense Oligonucleotide
aaccccatctttccaactcc

SEQ ID NO: 180 - DNA, 20 bp, Artificial Sequence
  Antisense Oligonucleotide
aagacttgatgcattatata

SEQ ID NO: 181 - DNA, 20 bp, Artificial Sequence
  Antisense Oligonucleotide
gacacagagagataagaaag

SEQ ID NO: 182 - DNA, 20 bp, Artificial Sequence
  Antisense Oligonucleotide
gagactcaagtgcaaataga

SEQ ID NO: 183 - DNA, 20 bp, Artificial Sequence
  Antisense Oligonucleotide
ttttacaggagctgaggtga

SEQ ID NO: 184 - DNA, 20 bp, Artificial Sequence
  Antisense Oligonucleotide
tgccactcttttacaggagc

SEQ ID NO: 185 - DNA, 20 bp, Artificial Sequence
  Antisense Oligonucleotide
atgttttttacttgccactc

SEQ ID NO: 186 - DNA, 20 bp, Artificial Sequence
  Antisense Oligonucleotide
tacaggagaatcagagcccc

SEQ ID NO: 187 - DNA, 20 bp, Artificial Sequence
  Antisense Oligonucleotide
gtatatttaattatcacaat

SEQ ID NO: 188 - DNA, 20 bp, Artificial Sequence
  Antisense Oligonucleotide
tctcaatgtcatgattgtgt

SEQ ID NO: 189 - DNA, 20 bp, Artificial Sequence
  Antisense Oligonucleotide
aagggtagaaatgcagttct

SEQ ID NO: 190 - DNA, 20 bp, Artificial Sequence
  Antisense Oligonucleotide
tgtatataccagtacttttt

SEQ ID NO: 191 - DNA, 20 bp, Artificial Sequence
  Antisense Oligonucleotide
gttttttttaaccctatttc

SEQ ID NO: 192 - DNA, 20 bp, Artificial Sequence
  Antisense Oligonucleotide
ctcatatagagagagcttga

SEQ ID NO: 193 - DNA, 20 bp, Artificial Sequence
  Antisense Oligonucleotide
taactctagtacattttggt

SEQ ID NO: 194 - DNA, 20 bp, Artificial Sequence
  Antisense Oligonucleotide
ggttttttatttcactaaac

SEQ ID NO: 195 - DNA, 20 bp, Artificial Sequence
  Antisense Oligonucleotide
ccatgcccggccagctgact

SEQ ID NO: 196 - DNA, 20 bp, Artificial Sequence
  Antisense Oligonucleotide
gggattacaagcatgagcca

SEQ ID NO: 197 - DNA, 20 bp, Artificial Sequence
  Antisense Oligonucleotide
cctcggcctcccaaagtgct

SEQ ID NO: 198 - DNA, 20 bp, Artificial Sequence
  Antisense Oligonucleotide
ctgacctcgtgatccacctg

SEQ ID NO: 199 - DNA, 20 bp, Artificial Sequence
  Antisense Oligonucleotide
actggtctcaaactcctgac

SEQ ID NO: 200 - DNA, 20 bp, Artificial Sequence
  Antisense Oligonucleotide
ccagactggtctcaaactcc

SEQ ID NO: 201 - DNA, 20 bp, Artificial Sequence
  Antisense Oligonucleotide
gacggggtttcaccatgttg

SEQ ID NO: 202 - DNA, 20 bp, Artificial Sequence
  Antisense Oligonucleotide
atttttgtatttttagtaca

SEQ ID NO: 203 - DNA, 20 bp, Artificial Sequence
  Antisense Oligonucleotide
cccaccaccacgcccagcta

SEQ ID NO: 204 - DNA, 20 bp, Artificial Sequence
  Antisense Oligonucleotide
agtagctgggattacaggca

SEQ ID NO: 205 - DNA, 20 bp, Artificial Sequence
  Antisense Oligonucleotide
gattctcctgcctcagcctc

SEQ ID NO: 206 - DNA, 20 bp, Artificial Sequence
  Antisense Oligonucleotide
acctcccgggttcaagcgat

SEQ ID NO: 207 - DNA, 20 bp, Artificial Sequence
  Antisense Oligonucleotide
ggctcactgccacctccacc

SEQ ID NO: 208 - DNA, 20 bp, Artificial Sequence
  Antisense Oligonucleotide
ctgcattgcagtggcgtgat

SEQ ID NO: 209 - DNA, 20 bp, Artificial Sequence
  Antisense Oligonucleotide
gtctagctctgttgcccggg

SEQ ID NO: 210 - DNA, 20 bp, Artificial Sequence
  Antisense Oligonucleotide
ttttttgttcttttgagaca

SEQ ID NO: 211 - DNA, 20 bp, Artificial Sequence
  Antisense Oligonucleotide
gagtttttttgtgttttttt

SEQ ID NO: 212 - DNA, 20 bp, Artificial Sequence
  Antisense Oligonucleotide
aattggttaagaagctgact

SEQ ID NO: 213 - DNA, 20 bp, Artificial Sequence
  Antisense Oligonucleotide
gccctggatgacacggaagc

SEQ ID NO: 214 - DNA, 20 bp, Artificial Sequence
  Antisense Oligonucleotide
ctcaatctgcacagaatggg

SEQ ID NO: 215 - DNA, 20 bp, Artificial Sequence
  Antisense Oligonucleotide
ccacctgtgtggtgcccaca

SEQ ID NO: 216 - DNA, 20 bp, Artificial Sequence
  Antisense Oligonucleotide
aggaagcactgaagcagcaa

SEQ ID NO: 217 - DNA, 20 bp, Artificial Sequence
  Antisense Oligonucleotide
caggcccaaggaaaaagagc

SEQ ID NO: 218 - DNA, 20 bp, Artificial Sequence
  Antisense Oligonucleotide
ttccctatggaacccagaag

SEQ ID NO: 219 - DNA, 20 bp, Artificial Sequence
  Antisense Oligonucleotide
gtgtctttctttcttactgt

SEQ ID NO: 220 - DNA, 20 bp, Artificial Sequence
  Antisense Oligonucleotide
tatgcatttatggtaaggat

SEQ ID NO: 221 - DNA, 20 bp, Artificial Sequence
  Antisense Oligonucleotide
ttctatttgtaggtggacca

SEQ ID NO: 222 - DNA, 20 bp, Artificial Sequence
  Antisense Oligonucleotide
ggcagatcatttaaatattt

SEQ ID NO: 223 - DNA, 20 bp, Artificial Sequence
  Antisense Oligonucleotide
agaatatcactttgtataaa

SEQ ID NO: 224 - DNA, 20 bp, Artificial Sequence
  Antisense Oligonucleotide
aggtaaattatcaaaggtag

SEQ ID NO: 225 - DNA, 20 bp, Artificial Sequence
  Antisense Oligonucleotide
gcagataaaaacatttaagc

SEQ ID NO: 226 - DNA, 20 bp, Artificial Sequence
  Antisense Oligonucleotide
tggatacagtactttgcagt

SEQ ID NO: 227 - DNA, 20 bp, Artificial Sequence
  Antisense Oligonucleotide
tggatgaggaaattttactt

SEQ ID NO: 228 - DNA, 20 bp, Artificial Sequence
  Antisense Oligonucleotide
caaaacagtttgaaagatat

SEQ ID NO: 229 - DNA, 20 bp, Artificial Sequence
  Antisense Oligonucleotide
aaatatatggcattagttaa

SEQ ID NO: 230 - DNA, 20 bp, Artificial Sequence
  Antisense Oligonucleotide
tcaagtgtgcagatacttac

SEQ ID NO: 231 - DNA, 20 bp, Artificial Sequence
  Antisense Oligonucleotide
aaccatctaacgttgctgta

SEQ ID NO: 232 - DNA, 20 bp, Artificial Sequence
  Antisense Oligonucleotide
cctttagggtttaccatcaa

SEQ ID NO: 233 - DNA, 20 bp, Artificial Sequence
  Antisense Oligonucleotide
atacacactcttggagtcct

SEQ ID NO: 234 - DNA, 20 bp, Artificial Sequence
  Antisense Oligonucleotide
tctgataaaactataaataa

SEQ ID NO: 235 - DNA, 20 bp, Artificial Sequence
  Antisense Oligonucleotide
gcattcaaataattgtcatc

SEQ ID NO: 236 - DNA, 20 bp, Artificial Sequence
  Antisense Oligonucleotide
gaaaggaatccatataattg

SEQ ID NO: 237 - DNA, 20 bp, Artificial Sequence
  Antisense Oligonucleotide
cccatcctccagcaaaaaat

SEQ ID NO: 238 - DNA, 20 bp, Artificial Sequence
  Antisense Oligonucleotide
tataaactttggtttcttct

SEQ ID NO: 239 - DNA, 20 bp, Artificial Sequence
  Antisense Oligonucleotide
gctttctcaatgtgaaggtc

SEQ ID NO: 240 - DNA, 20 bp, Artificial Sequence
  Antisense Oligonucleotide
agctgaagttcaaaactgaa

SEQ ID NO: 241 - DNA, 20 bp, Artificial Sequence
  Antisense Oligonucleotide
ctgttgtttttgaatctgat

SEQ ID NO: 242 - DNA, 20 bp, Artificial Sequence
  Antisense Oligonucleotide
taagaatgtcttggttcttt

SEQ ID NO: 243 - DNA, 20 bp, Artificial Sequence
  Antisense Oligonucleotide
agctgaaagtacaggcatct

SEQ ID NO: 244 - DNA, 20 bp, Artificial Sequence
  Antisense Oligonucleotide
gaactcatgaatttataccc

SEQ ID NO: 245 - DNA, 20 bp, Artificial Sequence
  Antisense Oligonucleotide
ttggtcaggtttcaatcttt

SEQ ID NO: 246 - DNA, 20 bp, Artificial Sequence
  Antisense Oligonucleotide
cttccatgaaataaagcaaa

SEQ ID NO: 247 - DNA, 20 bp, Artificial Sequence
  Antisense Oligonucleotide
aacacctttgtagatcactt

SEQ ID NO: 248 - DNA, 20 bp, Artificial Sequence
  Antisense Oligonucleotide
gctgttttccaaatggcaca

SEQ ID NO: 249 - DNA, 20 bp, Artificial Sequence
  Antisense Oligonucleotide
taaggcttgaacacatgcac

SEQ ID NO: 250 - DNA, 20 bp, Artificial Sequence
  Antisense Oligonucleotide
aaaatacgacatcgccaatc

SEQ ID NO: 251 - DNA, 20 bp, Artificial Sequence
  Antisense Oligonucleotide
tggcattgccacacgtgagg

SEQ ID NO: 252 - DNA, 20 bp, Artificial Sequence
  Antisense Oligonucleotide
cacaggtaaagtaaagcctt

SEQ ID NO: 253 - DNA, 20 bp, Artificial Sequence
  Antisense Oligonucleotide
taattcatatagtgtgtact

SEQ ID NO: 254 - DNA, 20 bp, Artificial Sequence
  Antisense Oligonucleotide
gattaaatgtacgttggaaa

SEQ ID NO: 255 - DNA, 20 bp, Artificial Sequence
  Antisense Oligonucleotide
gggaatttgtgacccttatt

SEQ ID NO: 256 - DNA, 20 bp, Artificial Sequence
  Antisense Oligonucleotide
tttattccagagattgattt

SEQ ID NO: 257 - DNA, 20 bp, Artificial Sequence
  Antisense Oligonucleotide
gcaatttaattacctctcta

SEQ ID NO: 258 - DNA, 4992 bp, Artificial Sequence
  Antisense Oligonucleotide
gaagactccagatataggatcactccatgccatcaagaaagttgatgctattgggcccatctcaagctgatcttggcacctctcatgctctgctctcttcaaccagacctctacattccattttggaagaagactaaaaatggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggagccaccaacgtctgtcacaggagttggaaagatggggtttatataatgcatcaagtcttctttcttatctctctgtgtctctatttgcacttgagtctctcacctcagctcctgtaaaagagtggcaagtaaaaaacatggggctctgattctcctgtaattgtgataattaaatatacacacaatcatgacattgagaagaactgcatttctacccttaaaaagtactggtatatacagaaatagggttaaaaaaaactcaagctctctctatatgagaccaaaatgtactagagttagtttagtgaaataaaaaaccagtcagctggccgggcatggtggctcatgcttgtaatcccagcactttgggaggccgaggcaggtggatcacgaggtcaggagtttgagaccagtctggccaacatggtgaaaccccgtctgtactaaaaatacaaaaattagctgggcgtggtggtgggtgcctgtaatcccagctacttgggaggctgaggcaggagaatcgcttgaacccgggaggtggaggtggcagtgagccgagatcacgccactgcaatgcagcccgggcaacagagctagactgtctcaaaagaacaaaaaaaaaaaaacacaaaaaaactcagtcagcttcttaaccaattgcttccgtgtcatccagggccccattctgtgcagattgagtgtgggcaccacacaggtggttgctgcttcagtgcttcctgctctttttccttgggcctgcttctgggttccatagggaaacagtaagaaagaaagacacatccttaccataaatgcatatggtccacctacaaatagaaaaatatttaaatgatctgcctttatacaaagtgatattctctacctttgataatttacctgcttaaatgtttttatctgcactgcaaagtactgtatccaaagtaaaatttcctcatccaatatctttcaaactgttttgttaactaatgccatatatttgtaagtatctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaatctctggaataaatagagaggtaattaaattgctggagccaacta

SEQ ID NO: 259 - DNA, 20 bp, Artificial Sequence
  Antisense Oligonucleotide
aatgcttgtctgtgcagtcc

SEQ ID NO: 260 - DNA, 20 bp, Artificial Sequence
  Antisense Oligonucleotide
ccagttctgttagattctcc

SEQ ID NO: 261 - DNA, 20 bp, Artificial Sequence
  Antisense Oligonucleotide
tggttgcctctgaactccag

SEQ ID NO: 262 - DNA, 20 bp, Artificial Sequence
  Antisense Oligonucleotide
ttgtgtgctcctggacctac